SEQUENCE LISTING (11 sequences)

SEQ ID NO: 1 - protein, 470 aa, Artificial Sequence
  source: /note=“Description of Artificial Sequence Synthetic polypeptide”
MGWSCIILFLVATATGVHSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, Artificial Sequence
  source: /note=“Description of Artificial Sequence Synthetic polypeptide”
MGWSCIILFLVATATGVHSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 219 aa, Artificial Sequence
  source: /note=“Description of Artificial Sequence Synthetic polypeptide”
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRADAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 5 aa, Artificial Sequence
  source: /note=“Description of Artificial Sequence Synthetic peptide”
SYWMH

SEQ ID NO: 5 - protein, 17 aa, Artificial Sequence
  source: /note=“Description of Artificial Sequence Synthetic peptide”
EIDPSESNTNYNQKFKG

SEQ ID NO: 6 - protein, 12 aa, Artificial Sequence
  source: /note=“Description of Artificial Sequence Synthetic peptide”
GGYDGWDYAIDY

SEQ ID NO: 7 - protein, 16 aa, Artificial Sequence
  source: /note=“Description of Artificial Sequence Synthetic peptide”
RSSQSLAKSYGNTYLS

SEQ ID NO: 8 - protein, 7 aa, Artificial Sequence
  source: /note=“Description of Artificial Sequence Synthetic peptide”
GISNRFS

SEQ ID NO: 9 - protein, 9 aa, Artificial Sequence
  source: /note=“Description of Artificial Sequence Synthetic peptide”
LQGTHQPYT

SEQ ID NO: 10 - protein, 111 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPQTFGQGKVEIK

SEQ ID NO: 11 - protein, 119 aa, Artificial Sequence
  source: /note=“Description of Artificial Sequence Synthetic polypeptide”
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGGYYGSGSNYWGQGTLVTVSS

(19) United States
(12) Patent Application Publication
(10) Pub. No.: US 2020/0179486 A1
(43) Pub. Date: Jun. 11, 2020

(54) METHOD OF TREATING PEDIATRIC DISORDERS

(71) Applicant: Millennium Pharmaceuticals, Inc., Cambridge, MA (US)

(72) Inventors: Maria Rosario, Cambridge, MA (US); Michael A. Shetzline, Cambridge, MA (US); William R. Treem, Cambridge, MA (US)

(73) Assignee: Millennium Pharmaceuticals, Inc., Cambridge, MA (US)

(21) Appl. No.: 16/608,895

(22) PCT Filed: Apr. 26, 2018
(86) PCT No.: PCT/US2018/029579

§ 371 Date: Oct. 28, 2019

Related U.S. Application Data
(60) Provisional application No. 62/492,031, filed on Apr. 28, 2017.

(51) Int. Cl. A61K 38/17; A61P 1/00

(52) U.S. Cl. CPC A61K 38/1777; A61K 9/0019; A61P 1/00

(57) ABSTRACT

The invention provides methods for treating pediatric inflammatory bowel disease patients using vedolizumab.

### RELATED APPLICATIONS

This application claims priority to U.S. Provisional Application No. 62/492,031 filed on Apr. 28, 2017. The entire contents of the foregoing application are incorporated herein by reference.

### SEQUENCE LISTING

The instant application contains a Sequence Listing which has been submitted electronically in ASCII format and is hereby incorporated by reference in its entirety. Said ASCII copy, created on Apr. 25, 2018, is named 079259-0839_SL.txt and is 12,557 bytes in size.

### BACKGROUND

The incidence of pediatric inflammatory bowel disease (IBD) appears to be increasing. According to the Crohn's and Colitis Foundation of American, approximately 1 million Americans have either ulcerative colitis or Crohn's disease, of which approximately 100,000 are younger than 21 years.

IBD, such as ulcerative colitis and Crohn's disease, for example, can be a debilitating and progressive disease involving inflammation of the gastrointestinal tract. While the symptoms of ulcerative colitis are similar in both the pediatric and adult populations, pediatric patients usually present with more extensive disease. For approximately 25% of IBD patients, the onset of disease occurs during childhood or adolescence.

IBD treatments have included anti-inflammatory drugs (such as, corticosteroids and sulfasalazine), immunosuppressive drugs (such as, 6-mercaptopurine, cyclosporine and azathioprine) and surgery (such as, colectomy). Podolsky, New Engl. J. Med., 325:928-937 (1991) and Podolsky, New Engl. J. Med., 325:1008-1016 (1991). As the disease progresses, treatment progresses into regimens that expose patients to progressive risk of side effects and loss of quality of life.

Integrin receptors are important for regulating both lymphocyte recirculation and recruitment to sites of inflammation (Carlos, T. M. and Harlan, J. M., Blood, 84:2068-2101 (1994)). The human α4β7 integrin has several ligands, one of which is the mucosal vascular addressin MAdCAM-1 (Berlin, C., et al., Cell 74: 185-195 (1993); Erle, D. J., et al., J. Immunol. 153:517-528 (1994)), which is expressed on high endothelial venules in mesenteric lymph nodes and Peyer's patches (Streeter, P. R., et al., Nature 331:41-46 (1998)). As such, the α4β7 integrin acts as a homing receptor that mediates lymphocyte migration to intestinal mucosal lymphoid tissue (Schweighoffer, T., et al., J. Immunol. 151: 717-729 (1993)).

Antibodies against human α4β7 integrin, such as murine monoclonal antibody Act-1 (mAb Act-1), interfere with α4β7 integrin binding to mucosal addressin cell adhesion molecule-1 (MAdCAM-1) present on high endothelial venules in mucosal lymph nodes. Act-1 was originally isolated by Lazarovits, A. I., et al., J. Immunol. 133:1857-1862 (1984), from mice immunized with human tetanus toxoid-specific T lymphocytes and was reported to be a mouse IgG1/κ antibody. Subsequent analysis of the antibody by Schweighoffer, T., et al., J. Immunol. 151:717-729 (1993) demonstrated that it can bind to a subset of human memory CD4+T lymphocytes which selectively express the α4β7 integrin. Entyvio™ vedolizumab, an anti-α₄β₇ integrin monoclonal antibody (mAb) with structural features derived from Act-1, is indicated for treating ulcerative colitis (UC) and Crohn's disease (CD). Studies reporting the activity of vedolizumab in treating these disorders (Feagen et al. NEJM 369:699-710 (2013) and Sandborn et al. NEJM 369:711-721 (2013)) showed varying levels of success depending on the disorder and nature of prior therapies.

Although growth failure is a common sequela of ulcerative colitis and Crohn's disease in the pediatric population, pediatric patients with Crohn's disease appear to be at twice the risk of growth failure compared to those with ulcerative colitis (Motil et al., Gastroenterology 105:681-691 (1993)). Nutritional therapy and surgical resection have been shown to improve growth, but there remains a clear need for more effective and less morbid treatment options for the pediatric patient population.

### SUMMARY OF THE INVENTION

The invention relates to the treatment of pediatric patients suffering from inflammatory bowel disease (IBD), e.g., Crohn's disease (CD) or ulcerative colitis (UC), and uses of an α4β7-integrin antagonist for relief of pediatric IBD symptoms. In one aspect, the pediatric patient has moderately to severely active UC or CD. In one aspect, the methods comprise administering an anti-integrin antibody, such as an anti-α4β7 antibody, such as vedolizumab.

In one aspect, the pediatric patient having inflammatory bowel disease has an inadequate response to, loss of response to, or intolerance of at least one of the following agents: corticosteroids, immunomodulators, and/or tumor necrosis factor-alpha (TNF-α) antagonist therapy.

In one aspect, the invention relates to a method for treating inflammatory bowel disease in a pediatric patient, comprising intravenously administering to a pediatric patient having IBD: a first dose of 100 mg of an antibody that has binding specificity for human α4β7 integrin, a second dose of 100 mg of the antibody two weeks after the first dose, and a third dose of 100 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2. The method may further comprise a fourth dose of 100 mg at 14 weeks after the first dose. The method may further comprise a fourth dose of 200 mg at 14 weeks after the first dose. The method may further comprise a fifth and subsequent dose of 100 mg every eight weeks after the fourth dose. The method, may further comprise a fifth and subsequent dose of 200 mg every eight weeks after the fourth dose. The heavy chain of the antibody may comprise amino acids 20 to 470 of SEQ ID NO:1, and the light chain of the antibody may comprise amino acids 20 to 238 of SEQ ID NO:2. Each dose may be intravenously administered as an infusion over about 120 minutes. The pediatric patient may weigh less than 30 kg. The inflammatory bowel disease may be moderately to severely active Crohn's disease. The inflammatory bowel disease may be moderately to severely active ulcerative colitis. The pediatric patient may have had a lack of an adequate response with, lost response to, or was intolerant to a TNFα antagonist. The pediatric patient may have had an inadequate response or loss of response to a corticosteroid. The pediatric patient may have had an inadequate response or loss of response to an immunomodulator. A clinical response may be achieved at week 14. The pediatric patient may achieve remission of the inflammatory bowel disease.

In another aspect, the invention relates to a method for treating inflammatory bowel disease in a pediatric patient, comprising intravenously administering to a pediatric patient having IBD: a first dose of 200 mg of an antibody that has binding specificity for human α4β7 integrin, a second dose of 200 mg of the antibody two weeks after the first dose, and a third dose of 200 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2. The method may further comprise a fourth dose of 200 mg at 14 weeks after the first dose. The method may further comprise a fifth and subsequent dose of 200 mg every eight weeks after the fourth dose. The heavy chain of the antibody may comprise amino acids 20 to 470 of SEQ ID NO:1, and the light chain of the antibody may comprise amino acids 20 to 238 of SEQ ID NO:2. Each dose may be intravenously administered as an infusion over about 120 minutes. The pediatric patient may weigh less than 30 kg. The inflammatory bowel disease may be moderately to severely active Crohn's disease. The inflammatory bowel disease may be moderately to severely active ulcerative colitis. The pediatric patient may have had a lack of an adequate response with, lost response to, or was intolerant to a TNFα antagonist. The pediatric patient may have had an inadequate response or loss of response to a corticosteroid. The pediatric patient may have had an inadequate response or loss of response to an immunomodulator. A clinical response may be achieved at week 14. The pediatric patient may achieve remission of the inflammatory bowel disease.

In another aspect, the invention relates to a method for treating inflammatory bowel disease in a pediatric patient, comprising intravenously administering to a pediatric patient having IBD: a first dose of 150 mg of an antibody that has binding specificity for human α4β7 integrin, a second dose of 150 mg of the antibody two weeks after the first dose, and a third dose of 150 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2. The method may further comprise a fourth dose of 150 mg at 14 weeks after the first dose. The method may further comprise a fourth dose of 300 mg at 14 weeks after the first dose. The method may further comprise a fifth and subsequent dose of 150 mg every eight weeks after the fourth dose. The method may further comprise a fifth and subsequent dose of 300 mg every eight weeks after the fourth dose. The heavy chain of the antibody may comprise amino acids 20 to 470 of SEQ ID NO:1, and the light chain of the antibody may comprise amino acids 20 to 238 of SEQ ID NO:2. Each dose may be intravenously administered as an infusion over about 30 minutes. The pediatric patient may weigh 30 kg or more. The inflammatory bowel disease may be moderately to severely active Crohn's disease. The inflammatory bowel disease may be moderately to severely active ulcerative colitis. The pediatric patient may have had a lack of an adequate response with, lost response to, or was intolerant to a TNFα antagonist. The pediatric patient may have had an inadequate response or loss of response to a corticosteroid. The pediatric patient may have had an inadequate response or loss of response to an immunomodulator. A clinical response may be achieved at week 14. The pediatric patient may achieve remission of the inflammatory bowel disease.

In another aspect, the invention relates to a method for treating inflammatory bowel disease in a pediatric patient, comprising intravenously administering to a pediatric patient having IBD: a first dose of 300 mg of an antibody that has binding specificity for human α4β7 integrin, a second dose of 300 mg of the antibody two weeks after the first dose, and a third dose of 300 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2. The method may further comprise a fourth dose of 300 mg at 14 weeks after the first dose. The method may further comprise a fifth and subsequent dose of 300 mg every eight weeks after the fourth dose. The heavy chain of the antibody may comprise amino acids 20 to 470 of SEQ ID NO:1, and the light chain of the antibody may comprise amino acids 20 to 238 of SEQ ID NO:2. Each dose may be intravenously administered as an infusion over about 30 minutes. The pediatric patient may weigh 30 kg or more. The inflammatory bowel disease may be moderately to severely active Crohn's disease. The inflammatory bowel disease may be moderately to severely active ulcerative colitis. The pediatric patient may have had a lack of an adequate response with, lost response to, or was intolerant to a TNFα antagonist. The pediatric patient may have had an inadequate response or loss of response to a corticosteroid. The pediatric patient may have had an inadequate response or loss of response to an immunomodulator. A clinical response may be achieved at week 14. The pediatric patient may achieve remission of the inflammatory bowel disease.

In another aspect, the invention relates to a method for treating inflammatory bowel disease in a pediatric patient, comprising intravenously administering to a pediatric patient having IBD: a first dose of 100 mg of an antibody that has binding specificity for human α4β7 integrin, a second dose of 100 mg of the antibody two weeks after the first dose, and a third dose of 100 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs: Light chain: CDR1 SEQ ID NO:7, CDR2 SEQ ID NO:8 and CDR3 SEQ ID NO:9; and Heavy chain: CDR1 SEQ ID NO:4, CDR2 SEQ ID NO:5 and CDR3 SEQ ID NO:6.

In another aspect, the invention relates to a method for treating inflammatory bowel disease in a pediatric patient, comprising intravenously administering to a pediatric patient having IBD: a first dose of 200 mg of an antibody that has binding specificity for human α4β7 integrin, a second dose of 200 mg of the antibody two weeks after the first dose, and a third dose of 200 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs: Light chain: CDR1 SEQ ID NO:7, CDR2 SEQ ID NO:8 and CDR3 SEQ ID NO:9; and Heavy chain: CDR1 SEQ ID NO:4, CDR2 SEQ ID NO:5 and CDR3 SEQ ID NO:6.

In another aspect, the invention relates to a method for treating inflammatory bowel disease in a pediatric patient, comprising intravenously administering to a pediatric patient having IBD: a first dose of 150 mg of an antibody that has binding specificity for human α4β7 integrin, a second dose of 150 mg of the antibody two weeks after the first dose, and a third dose of 150 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs: Light chain: CDR1 SEQ ID NO:7, CDR2 SEQ ID NO:8 and CDR3 SEQ ID NO:9; and Heavy chain: CDR1 SEQ ID NO:4, CDR2 SEQ ID NO:5 and CDR3 SEQ ID NO:6.

In another aspect, the invention relates to a method for treating inflammatory bowel disease in a pediatric patient, comprising intravenously administering to a pediatric patient having IBD: a first dose of 300 mg of an antibody that has binding specificity for human α4β7 integrin, a second dose of 300 mg of the antibody two weeks after the first dose, and a third dose of 300 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs: Light chain: CDR1 SEQ ID NO:7, CDR2 SEQ ID NO:8 and CDR3 SEQ ID NO:9; and Heavy chain: CDR1 SEQ ID NO:4, CDR2 SEQ ID NO:5 and CDR3 SEQ ID NO:6. Subsequent doses of the antibody may be administered subcutaneously. Each subcutaneous dose may be 108 mg of antibody. The subcutaneous dose may be administered every two or four weeks to a pediatric patient who weighs 30 kg or more. The subcutaneous dose may be administered every three weeks, every four weeks, every five weeks, every six weeks, every seven weeks, every eight weeks, every nine weeks or every ten weeks to a pediatric patient who weighs 10 kg to 30 kg.

In another aspect, the invention relates to a method for treating inflammatory bowel disease (IBD) in a pediatric patient, comprising intravenously administering to a pediatric patient having IBD: a first dose of 200 mg of an antibody that has binding specificity for human α4β7 integrin, a second dose of 200 mg of the antibody two weeks after the first dose, and subcutaneously administering a third dose of 108 mg of the antibody six weeks after the first dose and subsequent doses of 108 mg of the antibody every two, three or four weeks thereafter, wherein the antibody comprises an antigen binding region of nonhuman origin and at least a portion of an antibody of human origin, wherein the antibody has binding specificity for the α4β7 complex, wherein the antigen-binding region comprises the CDRs: Light chain: CDR SEQ ID NO:7, CDR2 SEQ ID NO:8 and CDR3 SEQ ID NO:9; and Heavy chain: CDR1 SEQ ID NO:4, CDR2 SEQ ID NO:5 and CDR3 SEQ ID NO:6.

In another aspect, the invention relates to a method for treating a pediatric cancer patient undergoing allogeneic hematopoietic stem cell transplantation (allo-HSCT), comprising intravenously administering to a pediatric patient the day before allo-HSCT a first dose of 200 mg of an antibody that has binding specificity for human α4β7 integrin, a second dose of 200 mg of the antibody two weeks after the first dose, and subcutaneously administering a third dose of 108 mg of the antibody six weeks after the first dose and subsequent doses of 108 mg of the antibody every two, three or four weeks thereafter, wherein the antibody comprises an antigen binding region of nonhuman origin and at least a portion of an antibody of human origin, wherein the antibody has binding specificity for the α4β7 complex, wherein the antigen-binding region comprises the CDRs: Light chain: CDR SEQ ID NO:7, CDR2 SEQ ID NO:8 and CDR3 SEQ ID NO:9; and Heavy chain: CDR1 SEQ ID NO:4, CDR2 SEQ ID NO:5 and CDR3 SEQ ID NO:6.

In another aspect, the invention relates to a method for treating a pediatric patient with a monogenic defect with IBD-like pathology, comprising intravenously administering to the pediatric patient: a first dose of 200 mg of an antibody that has binding specificity for human α4β7 integrin, a second dose of 200 mg of the antibody two weeks after the first dose, and a third dose of 200 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs: Light chain: CDR SEQ ID NO:7, CDR2 SEQ ID NO:8 and CDR3 SEQ ID NO:9; and Heavy chain: CDR1 SEQ ID NO:4, CDR2 SEQ ID NO:5 and CDR3 SEQ ID NO:6. The monogenic defect with IBD-like pathology may be glycogen storage disease type 1b, loss of function of IL10 and mutations in IL10 or IL10 receptors, X-linked lymphoproliferative syndrome 2, IPEX syndrome caused by mutations in the transcription factor FOXP3, or chronic granulomatous disease. The method may further comprise a subsequent dose of 200 mg every eight weeks thereafter. The method may further comprise a subsequent dose of 200 mg until the pediatric patient is 30 kg or greater.

In another aspect, the invention relates to a vial manufactured to deliver 200 mg of anti-a4b7 antibody for treating a pediatric patient.

Any one of the methods described herein comprising a dose of 100 mg, 200 mg or 150 mg may further comprise raising the dose to 300 mg after the pediatric patients weighs 30 kg or more.

The antibody used in the methods described herein may be a humanized antibody. The humanized antibody may comprise a heavy chain variable region sequence of amino acids 20 to 140 of SEQ ID NO:1, and a light chain variable region sequence of amino acids 20 to 131 of SEQ ID NO:2.

### BRIEF DESCRIPTION OF THE DRAWINGS

FIG. 1 shows a schematic of study design. The study includes a four-week screening period, a 22-week double-blind treatment period (with last dose at week 14 for all subjects). Following the four-week screening period, subjects weighing greater than or equal to 30 kg will be dosed at weeks 0, 2, 6, and 14 with either 300 mg or 150 mg of vedolizumab intravenously. Subjects weighing less than 30 kg will be dosed at weeks 0, 2, 6, and 14 with either 200 mg or 100 mg of vedolizumab intravenously. A nondosing visit may be scheduled anytime between days 16 and 42 for pharmacokinetic collection. Subjects who consent to participate in the open-label extension (OLE) study may be eligible for OLE study dosing after procedures have been completed at week 22 (visit 9). Subjects who do not enter the OLE study or withdraw before week 22 will also complete EP Visit (week 22) procedures and a final safety visit 18 weeks after their last dose of study drug. Subjects who withdraw before week 22 will also participate in a long-term follow-up safety survey by telephone six months after the last dose of study drug. Subjects will provide informed consent/pediatric assent for participation in OLE study on or after completing week 14 of study. Subjects who do not enter the OLE study will complete the final safety visit 18 weeks after their last dose of study drug and participate in a long-term follow-up safety survey by telephone six months after the last dose of study drug.

FIG. 2 is a schematic of a second study design. This study will begin after the week 22 visit for the study present in Example 1 and FIG. 1. Subjects receiving the low dose (150 mg for subjects 30 kg or more; 100 mg for subjects less than 30 kg) of vedolizumab IV may be escalated to the high dose (300 mg for subjects 30 kg or more; 200 mg for subjects less than 30 kg) at the discretion of the investigator if the subject demonstrates disease worsening by PUCAI/PCDAI at two consecutive visits. Subjects who have their dose increased based on nonresponse should be dosed based on the weight at the time of nonresponse in the Study of Example 1, FIG. 1.

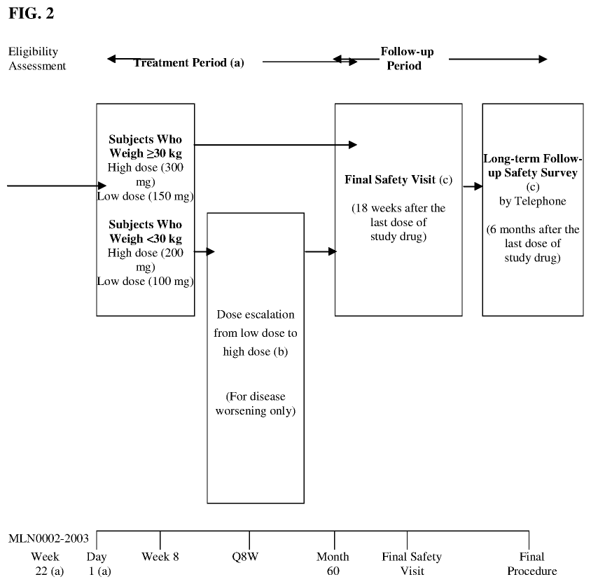

### DETAILED DESCRIPTION

The invention relates to methods for treating with an α4β7-integrin antagonist, such as an anti-α4β7 antibody, e.g., vedolizumab, a pediatric patient having inflammatory bowel disease (IBD), and methods for maintaining remission of IBD in a pediatric patient. The invention also relates to methods for treating with an α4β7-integrin antagonist, such as an anti-α4β7 antibody, e.g., vedolizumab, a pediatric patient at risk of or having graft versus host disease (GvHD), a pediatric patient having a monogenic defect with IBD-like pathology, a pediatric patient having glycogen storage disease type 1b, a pediatric patient having colitis related to loss of function of IL10 and mutations in IL10 or IL10 receptors, a pediatric patient having X-linked lymphoproliferative syndrome 2 (defect in the XIAP gene), a pediatric patient having IPEX syndrome caused by mutations in the transcription factor FOXP3, a pediatric patient having very early onset inflammatory bowel disease (onset <6 years of age), a pediatric patient having indeterminate colitis (IBDU) and a pediatric patient having chronic granulomatous disease-associated colitis.

The invention also relates to methods for treating with an α4β7-integrin antagonist, such as an anti-α4β7 antibody, e.g., vedolizumab, a pediatric patient having a monogenic defect with IBD-like pathology. The monogenic defect may be any one or combination of epithelial barrier and epithelial response defects (e.g., dystrophic epidermolysis bullosa, Kindler syndrome, X linked ectodermal dysplasia and immunodeficiency, ADAM-17 deficiency, familial diarrhea); neutropenia and defects in phagocyte bacterial killing (e.g., chronic granulomatous disease, glycogen storage disease type 1b, congenital neutropenia, leucocyte adhesion deficiency 1); hyper- and autoinflammatory disorders (e.g., mevalonate kinase deficiency, phospholipase Cγ2 defects, familial Mediterranean fever, familial haemophagocytic lymphohistiocytosis type 5, X linked lymphoproliferative syndrome 2, X linked lymphoproliferative syndrome 1, Hermansky-Pudlak syndrome); immune defects that include T and B cell selection and activation defects, B cell and antibody defects (e.g., common variable immunodeficiency type 1, common variable immunodeficiency type 8, agammaglobulinaemia, hyper-IgM syndrome, Wiskott-Aldrich syndrome, Omenn syndrome, Hyper IgE syndrome, trichohepatoenteric syndrome; PTEN hamaroma tumor syndrome, Hoyeraal Hreidarsson syndrome); regulatory T cells and immune regulation (e.g., X linked immune dysregulation, polyendocrinopathy, enteropathy, IL10 signalling defects); and defects in intestinal innervation (e.g., Hirschspring's disease).

Vedolizumab, a humanized monoclonal antibody that binds specifically to the α₄β₇ integrin, is indicated for the treatment of patients with moderately to severely active ulcerative colitis (UC) and Crohn's disease (CD). Vedolizumab has a novel gut-selective mechanism of action that differs from that of other currently marketed biologic agents for the treatment for inflammatory bowel disease (IBD), including natalizumab and tumor necrosis factor-α (TNF-α) antagonists. By binding to cell surface—expressed α₄β₇ integrin, vedolizumab blocks the interaction of a subset of memory gut-homing T lymphocytes with mucosal addressin cell adhesion molecule-1 (MAdCAM-1) expressed on endothelial cells. Consequently, migration of these cells into inflamed intestinal tissue is inhibited.

The efficacy and safety of vedolizumab induction and maintenance therapy were demonstrated in adult patients with UC in the GEMINI 1 trial (ClinicalTrials.gov number, NCT00783718) and in patients with CD in the GEMINI 2 (ClinicalTrials.gov number, NCT00783692) and GEMINI 3 (ClinicalTrials.gov number, NCT01224171) trials.

More recently, studies have been completed by various institutions around the world using vedolizumab to treat pediatric patients. In one study, the patients received vedolizumab intravenously at zero, two, and six weeks, and then approximately every eight weeks. The dose of vedolizumab was a fixed dose of 300 mg for 75% of the patients, but dosed by weight for the remaining smaller patients. Singh, et al., Inflamm. Bowel Dis., 22(9):2121-2126 (2016). In another study, pediatric inflammatory bowel disease was treated in a study including children aged 13 to 21 years old. Only the adult dose of 300 mg was administered at 0, 2, and 6 weeks followed by a maintenance phase at 8-week intervals. Patients weighing less than 40 kg were excluded from the study. Conrad, et al., Inflamm Bowel Dis., 22:2425-2431 (2016). Another study disclosed administering the adult dose of 300 mg to 81% of the children involved, while other children (weighing 28.5-48 kg) were administered a reduced dose (3.6-10.3 mg/kg). Ledder et al., J. of Crohn's and Colitis, 1230-1237 (2017). Thus, it is apparent that there is desire to expand the use of vedolizumab to treating pediatric patients. However, a need exists to develop a fixed dose that is suitable for smaller pediatric patients. Numerous dosing adjustments for a small patient, especially a very young patient in a phase of life known for rapid growth, is an unnecessary burden and an opportunity for mistakes to happen. A fixed pediatric dose for smaller patients is critical to simplify treatment of this patient population and avoid the potential for miscalculations based on weight.

### Definitions

A “Pediatric patient” as used herein, refers to a human patient up to the age of 18 years old.

As used herein, the “trough” serum concentration of an antibody refers to the concentration just before the next dose.

“Clinical remission” or “remission” as used herein with reference to ulcerative colitis subjects, refers to a complete Mayo score of less than or equal to 2 points and no individual subscore greater than 1 point. Crohn's disease “clinical remission” refers to a Crohn's Disease Activity Index (CDAI) score of 150 points or less or a HBI score of 4 or less. The CDAI score weighs factors including the number of liquid or very soft stools, the severity of abdominal pain, general well-being, extra-intestinal manifestations of the disease, such as arthritis, iritis, erythemia, fistula or abscess or fever, whether the patient is taking an antidiarrheal medication, abdominal mass, hematocrit and body weight. The “Harvey-Bradshaw Index” (HBI) is a simpler version of the CDAI for data collection purposes. It consists of only clinical parameters including general well-being, abdominal pain, number of liquid stools per day, abdominal mass, hematocrit, body weight, medications to control diarrhea and presence of complications, and requires only a single day's worth of diary entries.

Magnetic resonance enterography (MREn) is being evaluated as a method to measure remission.

“Endoscopic remission” as used herein, refers to a condition with a low endoscopic score. An example of a method to assess the endoscopic score in ulcerative colitis is flexible sigmoidoscopy. The endoscopic score in ulcerative colitis can be the Mayo subscore. An example of a method to assess the endoscopic score in Crohn's disease is ileocolonoscopy. The endoscopic score in Crohn's disease can be the simple endoscopic score for Crohn's Disease (SES-CD). The SES-CD can include measures such as the size of ulcers, the amount of ulcerated surface, the amount of affected surface and whether and to what extent the alimentary canal is narrowed.

A “clinical response” as used herein with reference to ulcerative colitis subjects refers to a reduction in complete Mayo score of 3 or greater points and 30% or greater from baseline, (or a partial Mayo score of 2 or greater points and 25% or greater from baseline, if the complete Mayo score was not performed at the visit) with an accompanying decrease in rectal bleeding subscore of 1 or greater points (≥1) or absolute rectal bleeding score of 1 or less point (≤1). A “clinical response” as used herein with reference to Crohn's disease subjects refers to a 70 point or greater decrease in CDAI score from baseline (week 0), a 50% or more reduction in SES-CD score from baseline or is a SES-CD score of 0 to 2 accompanied by a decrease in abdominal pain, or a 3 point or greater decrease from baseline HBI score. The terms “clinical response” and “response” e.g., alone without any adjective, are used interchangeably herein.

“Endoscopic response” as used herein, refers to a percentage decrease in an endoscopic score from baseline (e.g., at screening or just prior to initial dose). In Crohn's disease, endoscopic response can be assessed by a simple endoscopic score for Crohn's Disease (SES-CD).

“Baseline” as used herein describes a value of a parameter which is measured prior to the initial dose of a treatment. It can refer to a measurement of a sample obtained the same day, the day before, during the week before initial treatment, i.e., at a time period before the first dose when little change is expected until after the first dose and values of the measurement obtained after the first dose can be compared to this baseline value to represent the change caused by the dose.

“Mucosal healing” as used herein as used herein with reference to ulcerative colitis subjects, refers to a Mayo endoscopic subscore of less than or equal to 1. In reference to Crohn's disease, mucosal healing refers to an improvement in the amount or severity of wounding in mucosae, e.g., the digestive tract. For example, mucosal healing can refer to a decrease in the amount, size or severity of one or more than one ulcer in the digestive tract. In another example, mucosal healing refers to a decrease in one or more parameters selected from the group consisting of wall thickness, enhanced bowel wall contrast, mural edema, ulceration and perienteric vascularity. Such mucosal healing can be expressed as an SES-CD score, or a Magnetic Resonance Index of Activity (MaRIA) score. Complete mucosal healing in Crohn's disease includes absence of ulceration.

“PUCAI” or “Pediatric Ulcerative Colitis Activity Index” as used herein, refers to collection of 6 clinical items, including abdominal pain, rectal bleeding, stool consistency of most stools, number of stools per 24 hours, nocturnal stools (any episode causing wakening), and activity level. The PUCAI score ranges from 0 to 85; a score of less than ten denotes remission, 10 to 34 mild illness, 35 to 64 moderate disease, and 65 to 85 severe disease. A clinically significant response is defined as a PUCAI change of greater than or equal to 20.

“Clinical response based on PUCAI” as used herein, refers to a twenty point or greater decrease from baseline in Pediatric Ulcerative Colitis Activity Index (PUCAI) score. “Clinical remission based on PUCAI” as used herein refers to a PUCAI score of less than 10.

“Disease worsening” as used herein, refers to an increase in the PUCAI of greater than 20 points at two consecutive visits at least seven days apart, or the PUCAI was greater than 35 points at any scheduled or unscheduled visit (for ulcerative colitis subjects); or an increase in the PCDAI of greater than 15 points at two consecutive visits at least seven days apart, or the PCDAI was greater than 30 points at any scheduled or unscheduled visit.

“PCDAI” as used herein refers to an assessment specifically designed for use in children. The PCDAI includes a child-specific item: the height velocity variable as well as 3 laboratory parameters: hematocrit (adjusted for age and sex), ESR, and albumin level. The PCDAI score can range from 0-100, with higher scores signifying more active disease. A score of less than ten is consistent with inactive disease, 11 to 30 indicates mild disease, and greater than 30 is moderate to severe disease. A decrease of 12.5 points is taken as evidence of improvement. A clinical remission based on PDCAI is a PDCAI score of less than or equal to 10.

“European Quality of Life-5 Dimension (EQ-5D) visual analogue scale (VAS)” as used herein, refers to a questionnaire which is a validated (ahrq.gov/rice/eq5dproj.htm, “U.S. Valuation of the EuroQol EQ-SD™ Health States”, accessed 8 Aug. 2012, Bastida et al. BMC Gastroenterology 10:26-(2010), Konig et al. European Journal of Gastroenterology & Hepatology 14:1205-1215 (2002)) instrument used to measure general health-related quality of life (HRQOL) in patients and includes five domains—mobility, self-care, usual activities, pain/discomfort, and anxiety/depression. Patients choose the level of health problems they currently have on each item as “None”, “Moderate”, or “Extreme” and are scored a 1, 2, or 3, respectively. A composite EQ-5D score can be calculated from the individual scores to assess overall HRQOL. The EQ-5D Visual Analog Scale (VAS) score is a self-assigned rating of overall health using a 20 cm visual, vertical scale, with a score of 0 as the worst and 100 as best possible health. The EQ-5D and EQ-5D VAS have been shown in many studies to be valid and reliable instruments for measuring HRQOL in patients with GI diseases. A decrease of ≥0.3 points in the EQ-5D score represents a clinically meaningful improvement in HRQOL for patients. An increase of greater than or equal to 7 points in the EQ-5D VAS score represents a clinically meaningful improvement in HRQOL for patients.

The “Inflammatory Bowel Disease Questionnaire” ((IBDQ) questionnaire” (Irvine Journal of Pediatric Gastroenterology & Nutrition 28:S23-27 (1999)) is used to assess quality of life in adult patients with inflammatory bowel disease, ulcerative colitis, or Crohn's Disease and includes 32 questions on four areas of HRQOL: Bowel Systems (10 questions), Emotional Function (12 questions), Social Function (5 questions), and Systemic Function (5 questions). Patients are asked to recall symptoms and quality of life from the last 2 weeks and rate each item on a 7-point Likert scale (higher scores equate to higher quality of life). A total IBDQ score is calculated by summing the scores from each domain; the total IBDQ score ranges from 32 to 224. An IBDQ total score greater than 170 is characteristic of the health related quality of life (HRQoL) of patients in remission.

As used herein, “induction therapy” is an initial stage of therapy, wherein a patient is administered a relatively intensive dosing regimen of a therapeutic agent. The therapeutic agent, e.g., antibody, is administered in a way that quickly provides an effective amount of the agent suitable for certain purposes, such as inducing immune tolerance to the agent or for inducing a clinical response and ameliorating disease symptoms (see WO 2012/151247 and WO 2012/151248, incorporated herein by reference).

As used herein, “maintenance therapy” is after induction therapy and is administered in a way that continues the response achieved by induction therapy with a stable level of therapeutic agent, e.g., antibody. A maintenance regimen can prevent return of symptoms or relapse of disease, e.g., IBD (see WO 2012/151247 and WO 2012/151248, incorporated herein by reference). A maintenance regimen can provide convenience to the patient, e.g., be a simple dosing regimen or require infrequent trips for treatment.

The cell surface molecule, “α4β7 integrin,” or “α4β7,” is a heterodimer of an α₄ chain (CD49D, ITGA4) and a β₇ chain (ITGB7). Each chain can form a heterodimer with an alternative integrin chain, to form α₄β₁ or α_{E}β₇. Human α₄ and β₇ genes (GenBank (National Center for Biotechnology Information, Bethesda, Md.) RefSeq Accession numbers NM_000885 and NM_000889, respectively) are expressed by B and T lymphocytes, particularly memory CD4+ lymphocytes. Typical of many integrins, α4β7 can exist in either a resting or activated state. Ligands for α4β7 include vascular cell adhesion molecule (VCAM), fibronectin and mucosal addressin (MAdCAM (e.g., MAdCAM-1)). The α4β7 integrin mediates lymphocyte trafficking to GI mucosa and gut-associated lymphoid tissue (GALT) through adhesive interaction with mucosal addressin cell adhesion molecule-1 (MAdCAM-1), which is expressed on the endothelium of mesenteric lymph nodes and GI mucosa.

The term “antibody” herein is used in the broadest sense and specifically covers full length monoclonal antibodies, immunoglobulins, polyclonal antibodies, multispecific antibodies (e.g. bispecific antibodies) formed from at least two full length antibodies, e.g., each to a different antigen or epitope, and individual antigen binding fragments, including dAbs, scFv, Fab, F(ab)′₂, Fab′, including human, humanized and antibodies from non-human species and recombinant antigen binding forms such as monobodies and diabodies.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Antigen binding fragments” of an antibody comprise at least the variable regions of the heavy and/or light chains of an anti-α4β7 antibody. For example, an antigen binding fragment of vedolizumab comprises amino acid residues 20-131 of the humanized light chain sequence of SEQ ID NO:2. Examples of such antigen binding fragments include Fab fragments, Fab′ fragments, scFv and F(ab′)₂ fragments of a humanized antibody known in the art. Antigen binding fragments of the humanized antibody of the invention can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a recombinant construct encoding the heavy chain of an F(ab′)₂ fragment can be designed to include DNA sequences encoding the CH_{I} domain and hinge region of the heavy chain. In one aspect, antigen binding fragments inhibit binding of α4β7 integrin to one or more of its ligands (e.g. the mucosal addressin MAdCAM (e.g., MAdCAM-1), fibronectin).

The terms “Fc receptor” or “FcR” are used to describe a receptor that binds to the Fc region of an antibody. In one aspect, the FcR is a native sequence human FcR. In another aspect, the FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See review in M. Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:33-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)) and for regulating the persistence of immunoglobulin G (IgG) and albumin in the serum (reviewed by Rath et al., J. Clin. Immunol. 33 Suppl 1:S9-17 (2013)).

The term “hypervariable region” when used herein refers to the amino acid residues of an antibody which are responsible for antigen binding and are found in the “variable domain” of each chain. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein.

An “isolated” antibody is one which has been identified and separated and/or recovered from a component of its natural environment. In certain embodiments, the antibody will be purified (1) to greater than 95% by weight of protein as determined by the Lowry method, and alternatively, more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

“Treatment” refers to both therapeutic treatment and prophylactic or preventative measures. Those in need of treatment include those already with the disease as well as those in which the disease or its recurrence is to be prevented. Hence, the patient to be treated herein may have been diagnosed as having the disease or may be predisposed or susceptible to the disease. The terms “patient” and “subject” are used interchangeably herein.

The term “about” refers to following value may be the center point of a range, such as that is +/−5% of the value. If the value is a relative value given in percentages the term “about” also denotes that the thereafter following value may be no exact value, but is the center point of a range that is +/−5% of the value, whereby the upper limit of the range cannot exceed a value of 100%.

Treatment of Pediatric Inflammatory Bowel Disease Subjects with Anti-α4β7 Antibodies

In one aspect, the invention relates to methods of treating IBD (e.g., ulcerative colitis (UC), Crohn's disease (CD)) in a pediatric subject comprising administering to the pediatric subject an anti-α4β7 antibody described herein in an amount effective to treat IBD, e.g., in a child or adolescent. The pediatric patient or subject may be an adolescent or a child (e.g., 2 to 17 years old, inclusive). A pharmaceutical composition comprising an anti-α4β7 antibody can be used as described herein for treating IBD in a pediatric patient suffering therefrom. The pediatric patient may have moderately to severely active UC or CD. For example, the pediatric patient may have a complete Mayo score of 6 to 12 and a total of Mayo subscores of stool frequency and rectal bleeding ≥4 and an endoscopy subscore ≥2, or has moderately to severely active CD defined as simple endoscopic score for Crohn's disease (SES-CD) ≥7, and the Crohn's Disease Activity Index (CDAI) components of average daily Abdominal Pain Score of >1 for the 7 days prior, and total number of liquid/very soft stools >10 for the 7 days prior to the first dose of a treatment described herein. In some embodiments, the UC suffered by the pediatric patient is proximal to the rectum, e.g., pancolitis, not limited to proctitis. In some embodiments, the CD suffered by the pediatric patient involves the ileum and/or colon. In some embodiments, the pediatric patient also is suffering from structuring and disease penetration of the mucosae. The pediatric patient suffering from UC or CD may have growth failure.

In some embodiments, the pediatric patient suffering from CD has a mutation in the Nucleotide binding Oligomerization Domain containing 2 (NOD2/CARD15) gene (NCBI GeneID no. 64127, GenBank Accession No. of the longer isoform is NM_022162 and the shorter isoform is NM_01293557). In some embodiments, the pediatric patient suffering from CD has antineutrophil cytoplasmic antibody or anti-Saccharomyces cerevisiae antibody in the circulation.

In one aspect, the pediatric patient is 18 years of age or younger. In some embodiments, the pediatric patient is about 2 to about 17 years of age, about 2 to about 14 years of age, about 2 to about 10 years of age, about 2 to about 8 years of age, about 10 to about 18 years of age, about 8 to about 14 years of age, about 11 to about 15 years of age or about 13 to about 17 years of age.

The anti-α4β7 antibody for use in the methods or uses provided herein can bind to an epitope on the α4 chain (e.g., humanized MAb 21.6 (Bendig et al., U.S. Pat. No. 5,840,299), on the β7 chain (e.g., FIB504 or a humanized derivative (e.g., Fong et al., U.S. Pat. No. 7,528,236)), or to a combinatorial epitope formed by the association of the α4 chain with the β7 chain. In one aspect, the antibody is specific for the α4β7 integrin complex, e.g., binds a combinatorial epitope on the α4β7 complex, but does not bind an epitope on the α4 chain or the β7 chain unless the chains are in association with each other. The association of α4 integrin with β7 integrin can create a combinatorial epitope for example, by bringing into proximity residues present on both chains which together comprise the epitope or by conformationally exposing on one chain, e.g., the α4 integrin chain or the β7 integrin chain, an epitopic binding site that is inaccessible to antibody binding in the absence of the proper integrin partner or in the absence of integrin activation. In another aspect, the anti-α4β7 antibody binds both the α4 integrin chain and the β7 integrin chain, and thus, is specific for the α4β7 integrin complex. Combinatorial epitope anti-α4β7 antibodies can bind α4β7 but not bind α4β1, and/or not bind α_{Eβ}7, for example. In another aspect, the anti-α4β7 antibody binds to the same or substantially the same epitope as the Act-1 antibody (Lazarovits, A. I. et al., J. Immunol., 133(4): 1857-1862 (1984), Schweighoffer et al., J. Immunol., 151(2): 717-729, 1993; Bednarczyk et al., J. Biol. Chem., 269(11): 8348-8354, 1994). Murine ACT-1 Hybridoma cell line, which produces the murine Act-1 monoclonal antibody, was deposited under the provisions of the Budapest Treaty on Aug. 22, 2001, on behalf of Millennium Pharmaceuticals, Inc., 40 Landsdowne Street, Cambridge, Mass. 02139, U.S.A., at the American Type Culture Collection, 10801 University Boulevard, Manassas, Va. 20110-2209, U.S.A., under Accession No. PTA-3663. In another aspect, the anti-α4β7 antibody is a human antibody or an α4β7 binding protein using the CDRs provided in U.S. Patent Application Publication No. 2010/0254975.

In one aspect, the anti-α4β7 antibody inhibits binding of α4β7 to one or more of its ligands (e.g. the mucosal addressin, e.g., MAdCAM (e.g., MAdCAM-1), fibronectin, and/or vascular addressin (VCAM)). Primate MAdCAMs are described in the PCT publication WO 96/24673, the entire teachings of which are incorporated herein by this reference. In another aspect, the anti-α4β7 antibody inhibits binding of α4β7 to MAdCAM (e.g., MAdCAM-1) and/or fibronectin without inhibiting the binding of VCAM. In one aspect, the anti-integrin, e.g., an anti-α4β7 antibody, has the binding specificity, e.g., comprises the complementarity determining regions of the mouse Act-1 antibody. For example, an anti-α4β7 antibody comprises a heavy chain that contains the 3 heavy chain complementarity determining regions (CDRs, CDR1, SEQ ID NO:4, CDR2, SEQ ID NO:5 and CDR3, SEQ ID NO:6) of the mouse Act-1 antibody and suitable human heavy chain framework regions; and also comprises a light chain that contains the 3 light chain CDRs (CDR1, SEQ ID NO:7, CDR2, SEQ ID NO:8 and CDR3, SEQ ID NO:9) of the mouse Act-1 antibody and suitable human light chain framework regions. In some embodiments the anti-α4β7 antibody is an IgG1 isotype. In other embodiments, the anti-α4β7 antibody is an IgG2, IgG3, or IgG4 isotype.

In one aspect, the anti-α4β7 antibodies for use in the treatments are humanized versions of the mouse Act-1 antibody. Suitable methods for preparing humanized antibodies are well-known in the art. Generally, the humanized anti-α4β7 antibody will contain a heavy chain that contains the 3 heavy chain complementarity determining regions (CDRs, CDR1, SEQ ID NO:4, CDR2, SEQ ID NO:5 and CDR3, SEQ ID NO:6) of the mouse Act-1 antibody and suitable human heavy chain framework regions; and also contain a light chain that contains the 3 light chain CDRs (CDR1, SEQ ID NO:7, CDR2, SEQ ID NO:8 and CDR3, SEQ ID NO:9) of the mouse Act-1 antibody and suitable human light chain framework regions. The humanized Act-1 antibody can contain any suitable human framework regions, including consensus framework regions, with or without amino acid substitutions. For example, one or more of the framework amino acids can be replaced with another amino acid, such as the amino acid at the corresponding position in the mouse Act-1 antibody. The human constant region or portion thereof, if present, can be derived from the κ or λ, light chains, and/or the γ (e.g., γ1, γ2, γ3, γ4), μ, α (e.g., α1, α2), δ or ε heavy chains of human antibodies, including allelic variants. A particular constant region (e.g., IgG1), variant or portions thereof can be selected in order to tailor effector function. For example, a mutated constant region (variant) can be incorporated into a fusion protein to minimize binding to Fc receptors and/or ability to fix complement (see e.g., Winter et al., GB 2,209,757 B; Morrison et al., WO 89/07142; Morgan et al., WO 94/29351, Dec. 22, 1994). Humanized versions of Act-1 antibody were described in PCT publications nos. WO98/06248 and WO07/61679, the entire teachings of each of which are incorporated herein by this reference.

In one aspect, the anti-α4β7 antibody is vedolizumab. Vedolizumab (also called MLN0002, ENTYVIO™ or KYNTELES™) is a humanized immunoglobulin (Ig) G1 mAb directed against the human lymphocyte integrin α4β7. Vedolizumab binds the α4β7 integrin, antagonizes its adherence to MAdCAM-1 and as such, impairs the migration of gut homing leukocytes into GI mucosa. Vedolizumab is an integrin receptor antagonist indicated for adult patients with moderately to severely active UC or CD who have had an inadequate response with, lost response to, or were intolerant to a tumor necrosis factor (TNF) blocker or immunomodulator, or had an inadequate response with, were intolerant to, or demonstrated dependence on corticosteroids. For UC, vedolizumab is for inducing and maintaining clinical response, inducing and maintaining clinical remission, improving endoscopic appearance of the mucosa, and/or achieving corticosteroid-free remission. For CD, vedolizumab is for achieving clinical response, achieving clinical remission, and/or achieving corticosteroid-free remission. In some embodiments, corticosteroid-free remission is achieved through a tapering regimen during continued treatment with vedolizumab.

In another aspect, the humanized anti-α4β7 antibody for use in the treatment comprises a heavy chain variable region comprising amino acids 20 to 140 of SEQ ID NO:1, and a light chain variable region comprising amino acids 20 to 131 of SEQ ID NO:2 or amino acids 21 to 132 of SEQ ID NO:3. If desired, a suitable human constant region(s) can be present. For example, the humanized anti-α4β7 antibody can comprise a heavy chain that comprises amino acids 20 to 470 of SEQ ID NO:1 and a light chain comprising amino acids 21 to 239 of SEQ ID NO:3. In another example, the humanized anti-α4β7 antibody can comprise a heavy chain that comprises amino acids 20 to 470 of SEQ ID NO:1 and a light chain comprising amino acids 20 to 238 of SEQ ID NO:2. The humanized light chain of vedolizumab (e.g., Chemical Abstract Service (CAS, American Chemical Society) Registry number 943609-66-3), with two mouse residues switched for human residues, is more human than the light chain of LDP-02, another humanized anti-α4β7 antibody. In addition, LDP-02 has the somewhat hydrophobic, flexible alanine 114 and a hydrophilic site (Aspartate 115) that is replaced in vedolizumab with the slightly hydrophilic hydroxyl-containing threonine 114 and hydrophobic, potentially inward facing valine 115 residue.

Further substitutions to the humanized anti-α4β7 antibody sequence can be, for example, mutations to the heavy and light chain framework regions, such as a mutation of isoleucine to valine on residue 2 of SEQ ID NO:10; a mutation of methionine to valine on residue 4 of SEQ ID NO:10; a mutation of alanine to glycine on residue 24 of SEQ ID NO:11; a mutation of arginine to lysine at residue 38 of SEQ ID NO:11; a mutation of alanine to arginine at residue 40 of SEQ ID NO:11; a mutation of methionine to isoleucine on residue 48 of SEQ ID NO:11; a mutation of isoleucine to leucine on residue 69 of SEQ ID NO:11; a mutation of arginine to valine on residue 71 of SEQ ID NO:11; a mutation of threonine to isoleucine on residue 73 of SEQ ID NO:11; or any combination thereof; and replacement of the heavy chain CDRs with the CDRs (CDR1, SEQ ID NO:4, CDR2, SEQ ID NO:5 and CDR3, SEQ ID NO:6) of the mouse Act-1 antibody; and replacement of the light chain CDRs with the light chain CDRs (CDR1, SEQ ID NO:7, CDR2, SEQ ID NO:8 and CDR3, SEQ ID NO:9) of the mouse Act-1 antibody.

In one aspect, the humanized anti-α4β7 antibody for use in the treatment of a pediatric human patient is included in a stable formulation comprising a mixture of a non-reducing sugar, an anti-α4β7 antibody and at least one free amino acid (i.e., not attached to a protein), and the molar ratio of non-reducing sugar to anti-α4β7 antibody (mole:mole) is greater than 650:1. The formulation may be a liquid formulation or a dry formulation (e.g., lyophilized). The formulation can also contain a buffering agent. In some embodiments, the non-reducing sugar is mannitol, sorbitol, sucrose, trehalose, or any combination thereof.

In some embodiments, the free amino acid of the formulation is histidine, alanine, arginine, glycine, glutamic acid, or any combination thereof. The formulation can comprise between about 50 mM to about 175 mM of free amino acid. The formulation can comprise between about 100 mM and about 175 mM of free amino acid. The ratio of free amino acid to antibody molar ratio can be at least 250:1, or 200:1 to 500:1, or 250:1 to 400:1.

The formulation can also contain a surfactant. The surfactant can be polysorbate 20, polysorbate 80, a poloxamer, or any combination thereof. The surfactant may have a concentration of about 0.2 mg/ml to 2.5 mg/ml, about 0.4 mg/ml to 0.9 mg/ml, about 0.5 mg/ml to 0.8 mg/ml, about 1.8 mg/ml to 2.2 mg/ml. In some embodiments, the surfactant concentration is about 0.6 mg/ml. In some embodiments, the surfactant concentration is about 0.75 mg/ml. In some embodiments, the surfactant concentration is about 2.0 mg/ml.

In some aspects, the formulation can minimize immunogenicity of the anti-α4β7 antibody.

The formulation, e.g., in the dried state, can be stable for at least three months at 40° C., 75% relative humidity (RH). In the dried state, the lyophilized formulation has about 0.5% to 10%, about 0.8% to 7.5%, about 1% to 5%, ≤5%, ≤4%, ≤3% or ≤2.5% moisture, e.g., as determined by Karl Fisher analysis. Upon reconstitution, e.g., after storage at 25° C., 30° C. or 2-8° C., a stable lyophilized formulation comprises about 0%-10% aggregated anti-α4β7 antibody (e.g., dimers, trimers or multimeric forms of antibody and/or antibody degradation products, as measured by size exclusion chromatography). In some embodiments, the stored, reconstituted lyophilized formulation of anti-α4β7 antibody comprises about 0% to 5.0%, 0% to 2%, ≤2%, ≤1% or ≤0.5% aggregates.

In another aspect, the formulation is lyophilized and comprises at least about 5% to about 10% w/v anti-α4β7 antibody before lyophilization. The formulation can contain at least about 6% w/v anti-α4β7 antibody before lyophilization. The formulation can be reconstituted from a lyophilized formulation (e.g., reconstituted to comprise a stable liquid formulation). The dried formulation of an anti-α4β7 antibody comprises about 25% to 35% w/w or about 29% to 32% w/w anti-α4β7 antibody. The dried formulation of an anti-α4β7 antibody may further comprise about 30% to 65% w/w, about 40% to 60%, about 45% to 55%, or 50% to 52% w/w anti-α4β7 non-reducing sugar, such as sucrose or trehalose. The dried formulation of an anti-α4β7 antibody may further comprise about 5% to 20% w/w or about 10% to 15% w/w amino acid salt, such as arginine hydrochloride. The dried formulation may further comprise about 1% to 10% w/w, about 2% to 7% w/w, or about 4% to 6% w/w buffer, e.g., histidine. In some embodiments, the dried formulation comprises about 30% to 31% w/w anti-α4β7 antibody, e.g., vedolizumab, about 50% to 52% w/w sucrose, and about 12% to 14% w/w arginine hydrochloride. The above dried formulations may further comprise about 0.25% to 0.4% w/w, or about 0.9% to 1.2% w/w of polysorbate 80.

In another aspect, the invention relates to treating a pediatric patient with a stable formulation comprising a mixture of a non-reducing sugar, an anti-α4β7 antibody and at least one free amino acid, and the molar ratio of non-reducing sugar to anti-α4β7 antibody (mole:mole) is greater than 650:1 and the ratio of free amino acid to anti-α4β7 antibody (mole:mole) is greater than 250:1.

In another aspect, the invention relates to treating a pediatric patient with a stable formulation comprising a mixture of a non-reducing sugar, an anti-α4β7 antibody and at least one free amino acid, and the molar ratio of non-reducing sugar to anti-α4β7 antibody (mole:mole) is greater than 650:1 and the ratio of free amino acid to anti-α4β7 antibody (mole:mole) is greater than 250:1.

In another aspect, the invention relates to treating a pediatric patient with a stable liquid formulation, e.g., before lyophilization or after reconstitution with a solvent, comprising in aqueous solution with a non-reducing sugar, an anti-α4β7 antibody and at least one free amino acid, wherein the molar ratio of non-reducing sugar to anti-α4β7 antibody (mole:mole) is greater than 650:1. In yet a further aspect, the invention concerns a liquid formulation comprising at least about 40 mg/ml to about 80 mg/ml anti-α4β7 antibody, at least about 50-175 mM of one or more amino acids, and at least about 6% to at least about 11% (w/v) sugar. The liquid formulation may also contain a buffering agent. A buffering agent may be histidine, succinate, phosphate, glycine or citrate. In some embodiments the liquid formulation also comprises a metal chelator. In some embodiments, the liquid formulation also comprises an anti-oxidant, such as citrate. In some embodiments, the citrate concentration is about 5 mM to 40 mM, about 7 mM to 10 mM, or about 20 to 30 mM. In some embodiments, the citrate concentration is about 25 mM. In some embodiments, the citrate concentration is about 9.4 mM.

In another aspect, the invention relates to treating a pediatric patient with a liquid formulation comprising at least about 60 mg/ml anti-α4β7 antibody, at least about 10% (w/v) non-reducing sugar, and at least about 125 mM of one or more free amino acids. In some embodiments, the liquid formulation is about 60 mg/ml anti-α4β7 antibody.

In another aspect, the invention relates to treating a pediatric patient with a liquid formulation comprising at least about 60 mg/ml anti-α4β7 antibody, at least about 10% (w/v) non-reducing sugar, and at least about 175 mM of one or more free amino acids.

In still yet a further aspect, the invention also relates to treating a pediatric patient with a dry, e.g., lyophilized formulation comprising a mixture of a non-reducing sugar, an anti-α4β7 antibody, histidine, arginine, and polysorbate 80, and the molar ratio of non-reducing sugar to anti-α4β7 antibody (mole:mole) is greater than 650:1.

In still yet a further aspect, the invention relates to treating a pediatric patient with a lyophilized formulation comprising a mixture of a non-reducing sugar, an anti-α4β7 antibody, histidine, arginine, and polysorbate 80. In this aspect, the molar ratio of non-reducing sugar to anti-α4β7 antibody (mole:mole) is greater than 650:1. Furthermore, the molar ratio of arginine to anti-α4β7 antibody (mole:mole) in the formulation is greater than 250:1 or the molar ratio of histidine and arginine to antibody (mole:mole) is about 200:1 to about 500:1.

In another aspect, the invention relates to treating a pediatric patient with a stable liquid pharmaceutical formulation comprising a mixture of anti-α4β7 antibody, citrate, histidine, arginine and polysorbate 80. The formulation can be present in a container, such as a vial, cartridge, syringe or autoinjector. In some embodiments, the liquid formulation comprises at least about 120 mg/ml anti-α4β7 antibody, at least about 140 mg/ml anti-α4β7 antibody, 140 mg/ml to 250 mg/ml anti-α4β7 antibody, 140 mg/ml to 175 mg/ml anti-α4β7 antibody or 150 mg/ml to 170 mg/ml anti-α4β7 antibody. In other embodiments, the liquid formulation is about 160 mg/ml anti-α4β7 antibody.

In one aspect, the humanized anti-α4β7 antibody for use in the treatment of a pediatric patient is lyophilized and stored as a single dose in one container, e.g., a vial. The container, e.g., vial is stored refrigerated, e.g., at about 2-8° C., or at room temperature, e.g., at about 20° C. to 35° C., about 25° C. or about 30° C., until it is administered to a subject in need thereof. A vial may for example be a 10, 20 or 50 cc vial (for example for a 60 mg/ml dose). The container, e.g., vial may contain about 90 to 115 mg, about 95 to 105 mg, at least about 100 mg, about 135 to 160 mg, about 145 to 155 mg, at least about 150 mg, about 180 to 220 mg, about 190 to 210 mg, about 195 to 205 mg, at least about 200 mg, about 280 mg to 320 mg, about 290 mg to 310 mg, at least about 300 mg, about 380 to 420 mg, about 390 to 410 mg, at least about 400 mg, about 580 to 620 mg, about 590 to 610 mg, or at least about 600 mg of anti-α4β7 antibody. In one aspect, the vial contains about 200 mg of anti-α4β7 antibody. The vial may contain enough of the anti-α4β7 antibody, e.g., vedolizumab, to permit delivery of, e.g., be manufactured to deliver, about 100 mg, about 150 mg, about 200 mg, about 300 mg, about 400 mg, or about 600 mg of anti-α4β7 antibody. For example, the vial may contain about 15%, about 12%, about 10% or about 8% more anti-α4β7 antibody than the dose amount.

In another aspect, the anti-α4β7 antibody, e.g., vedolizumab, for use in the treatment of a pediatric patient is in a stable liquid pharmaceutical composition stored in a container, e.g., a vial, a syringe or cartridge, at about 2-8° C. until it is administered to a subject in need thereof. The syringe or cartridge may be a 1 mL or 2 mL container (for example for a 160 mg/mL dose) or more than 2 ml, e.g., for a higher dose (at least 320 mg or 400 mg or higher). The syringe or cartridge may contain at least about 20 mg, at least about 50 mg, at least about 70 mg, at least about 80 mg, at least about 100 mg, at least about 108 mg, at least about 120 mg, at least about 155 mg, at least about 180 mg, at least about 200 mg, at least about 240 mg, at least about 300 mg, at least about 360 mg, at least about 400 mg, or at least about 500 mg of anti-α4β7 antibody. In some embodiments, the container, e.g., syringe or cartridge may be manufactured to deliver about 20 to 120 mg, about 40 mg to 70 mg, about 45 to 65 mg, about 50 to 57 mg or about 54 mg of anti-α4β7 antibody, e.g., vedolizumab. In other embodiments, the syringe or cartridge may be manufactured to deliver about 90 to 120 mg, about 95 to 115 mg, about 100 to 112 mg or about 108 mg of anti-α4β7 antibody, e.g., vedolizumab. In other embodiments, the syringe or cartridge may be manufactured to deliver about 140 to 250 mg, about 150 to 200 mg, about 160 to 170 mg, about 160 to 250 mg, about 175 mg to 210 mg or about 160 mg, about 165 mg, about 180 mg or about 200 mg of anti-α4β7 antibody, e.g., vedolizumab.

The present invention provides, in a first aspect, a method for treating a pediatric patient having inflammatory bowel disease (IBD) with an anti-α4β7 antibody, e.g., vedolizumab. In this aspect, the method comprises administering an intravenous dose of vedolizumab. The dose may be 100 mg, 150 mg, 200 mg, or 300 mg anti-α4β7 antibody. In some embodiments, the dose will be selected based on the weight of the patient. In one aspect, the pediatric patient weighs 30 kg or greater. In another aspect, the pediatric patient weighs less than 30 kg. In some embodiments, the pediatric patient who weighs 30 kg or greater weighs about 30 to 35 kg, about 30 to 40 kg, about 35 to 45 kg, about 40 to 45 kg, about 30 to 50 kg, or about 40 to 50 kg. In other embodiments, the pediatric patient who weighs less than 30 kg weighs about 5 kg to 30 kg, about 10 kg to 15 kg, about 15 kg to 20 kg, about 10 kg to 20 kg, about 12 kg to 22 kg, about 10 to 25 kg, about 15 to 30 kg or about 10 kg to 30 kg.

In some embodiments, a pediatric patient weighing less than 30 kg may be administered a dose of 100 mg or 200 mg of anti-α4β7 antibody. In some embodiments, a pediatric patient weighing 30 kg or more may be administered a dose of 150 mg or 300 mg anti-α4β7 antibody.

An anti-α4β7 antibody, is administered in an effective amount which inhibits binding of α4β7 integrin to a ligand thereof. For therapy, an effective amount will be sufficient to achieve the desired effect of response or remission (e.g., as defined herein). An α4β7 antagonist, such as an anti-α4β7 antibody may be administered in a unit dose or multiple doses. Examples of modes of administration include topical routes such as nasal or inhalational or transdermal administration, enteral routes, such as through a feeding tube or suppository, and parenteral routes, such as intravenous, intramuscular, subcutaneous, intraarterial, intraperitoneal, or intravitreal administration. Suitable dosages for antibodies can be from about 0.1 mg/kg body weight to about 10.0 mg/kg body weight, about 1 mg/kg to about 60 mg/kg body weight, about 5 mg/kg to about 30 mg/kg body weight, about 6.5 mg/kg to about 20 mg/kg body weight, or at least 15 mg/kg or at least 20 mg/kg body weight per treatment.

It is surprising that administration of a fixed dose of 100 mg, 150 mg, or 200 mg, e.g., from a dosage form, e.g., a vial, manufactured to deliver about 95 to 110 mg, 100 mg, 108 mg, 145 mg to 155 mg, 150 mg, 155 mg to 170 mg, 190 to 210 mg or 200 mg of an anti-α4β7 antibody, e.g., vedolizumab, to a small pediatric patient, e.g., 5 kg to 35 kg, 10 kg to 30 kg, or less than 30 kg, is safe. In these embodiments, the smallest patients may be administered at least 20 mg/kg anti-α4β7 antibody, a dose level unprecedented in therapeutic use of anti-α4β7 antibody, e.g., vedolizumab, wherein the smallest adults are administered about 5 to 7 mg/kg anti-α4β7 antibody from a 300 mg dosage form. However, the juvenile monkey study showed the safety of anti-α4β7 antibody, e.g., vedolizumab, at doses up to 100 mg/kg.

In some embodiments, the anti-α4β7 antibody, such as vedolizumab is provided as a dry, lyophilized formulation which can be reconstituted with a liquid, such as sterile water, for administration. Administration of a reconstituted formulation can be by parenteral injection by one of the routes described above. An intravenous injection can be by infusion, such as by further dilution with sterile isotonic saline, buffer, e.g., phosphate-buffered saline or Ringer's (lactated or dextrose) solution. In some embodiments, the anti-α4β7 antibody is administered by subcutaneous injection, e.g., a dose of about 54 mg, 108 mg or about 165 mg or about 216 mg, at about every two, three or four weeks after the start of therapy or after the third subsequent dose.

In some embodiments, vedolizumab is administered by one or more of intravenous injection, subcutaneous injection, or infusion. In some embodiments, vedolizumab is administered at a dose of 40 mg, 50 mg, 60 mg, 70 mg, 75 mg, 80 mg, 90 mg, 100 mg, 120 mg, 125 mg, 150 mg, 200 mg, 300 mg, 450 mg, 600 mg, 45-125 mg, 80-120 mg, 125-250 mg, or 90-210 mg. In some embodiments, the vedolizumab is administered, for example subcutaneously, at a dose of 0.5 mg/kg, 1.0 mg/kg, 1.5 mg/kg, 2.0 mg/kg, 2.5 mg/kg, 3.0 mg/kg. 4.0 mg/kg, or 5.0 mg/kg, at a dose of 54 mg, 108 mg, 216 mg, 160 mg, 165 mg, 320 mg, or 480 mg. The vedolizumab may be administered once per day, per week, per month, or per year. A vedolizumab dosing regimen may have an initial or induction phase and a maintenance phase. An induction phase may be one or more than one, e.g., two, three or four doses, of high amounts or without long times, such as only one week, two weeks, three weeks or four weeks between each dose. For example, an induction regimen may have two doses, one at day (week) zero and one at week 2 (day 14). A maintenance phase, e.g., to maintain remission of the IBD, may have lower doses or doses further apart than in the induction phase. In some embodiments, the maintenance dosing is every 4 weeks, every 6 weeks, every 8 weeks, every 10 weeks, or every 12 weeks. In some embodiments, the vedolizumab is administered at zero, two and six weeks (induction), and then every four weeks or every eight weeks thereafter (maintenance). Pediatric patients with IBD refractory to other therapies may need longer induction periods, e.g., 8, 10, 12 or 14 weeks, before beginning maintenance therapy.

In one embodiment, vedolizumab is administered intravenously at zero, two, and six weeks, and then subjects who do not achieve clinical response (based on PUCAI/PCDAI) at week 14 will receive a double dose at week 14 (e.g., a patient receiving 100 mg doses at weeks 0, 2, and 6, who does not achieve clinical response at week 14 will be administered a 200 mg dose at week 14; a patient receiving 150 mg doses at weeks 0, 2, and 6, who does not achieve a clinical response at week 14 will be administered a 300 mg dose at week 14).

In an embodiment, vedolizumab is administered intravenously at zero, two, six weeks, and 14 weeks. In some embodiments, vedolizumab is administered intravenously at zero, two, six, and 14 weeks, then every four or eight weeks thereafter. In some embodiments, vedolizumab is administered intravenously at zero, two, six, ten, and 14 weeks, then every four or eight weeks thereafter. In some embodiments, vedolizumab is administered one or more times, and then at least one month, at least six months, or at least one year later, vedolizumab is again administered one or more times.

In some embodiments, 100 or 150 mg vedolizumab may be administered by intravenous infusion at zero, two weeks, six weeks, fourteen weeks, and then, at eight week intervals thereafter, 200 or 300 mg, respectively (i.e., twice the prior dose) of vedolizumab may be administered intravenously. In some embodiments, 100 or 150 mg vedolizumab may be administered by intravenous infusion at zero, two weeks, and at six weeks, and then, at four week intervals or eight week intervals thereafter, 200 or 300 mg, respectively (i.e., twice the prior dose) of vedolizumab may be administered intravenously. In some embodiments, 100 or 150 mg vedolizumab may be administered by intravenous infusion at zero and two weeks, and then at six weeks, 200 or 300 mg, respectively (i.e., twice the prior dose) vedolizumab may be administered by intravenous infusion, and then at four week intervals or eight week intervals thereafter, 200 or 300 mg of vedolizumab may be administered intravenously. In some embodiments, if the pediatric patient is treated with vedolizumab on weeks zero, 2, 6 and 14 at a dose based on a weight of less than 30 kg, and during treatment, grows to be 30 kg or greater, then the pediatric patient may be treated at a dose based on the higher weight.

In some embodiments, a pediatric patient being treated with the low dose relative to size (150 mg for subjects 30 kg or more; 100 mg for subjects less than 30 kg) of the anti-α4β7 antibody may be escalated to receive the higher dose relative to size (300 mg for subjects 30 kg or more; 200 mg for subjects less than 30 kg) if the patient demonstrates disease worsening.

In some embodiments, 200 or 300 mg vedolizumab may be administered by intravenous infusion at zero and two weeks, 200 or 300 mg vedolizumab may be administered by intravenous infusion at six weeks, and then at two-, three- or four-week intervals thereafter, vedolizumab may be administered subcutaneously, e.g., at a dose of 54, 108, 165 or 216 mg. In some embodiments, 100 or 150 mg vedolizumab may be administered by intravenous infusion at zero and two weeks, 200 or 300 mg vedolizumab may be administered by intravenous infusion at six weeks and at 14 weeks, and then at two-, three- or four-week intervals, thereafter, vedolizumab may be administered subcutaneously, e.g., at a dose of 54, 108, 165 or 216 mg. In some embodiments, 100 or 150 mg vedolizumab may be administered by intravenous infusion at zero and two weeks, 200 or 300 mg vedolizumab may be administered by intravenous infusion at six weeks, and then at two-, three- or four-week intervals thereafter, vedolizumab may be administered subcutaneously, e.g., at a dose of 54, 108, 165 or 216 mg.

In some embodiments, 100 or 200 mg vedolizumab may be administered by intravenous infusion to a patient weighing less than 30 kg, or 10 kg to less than 30 kg at zero and two weeks, 100 or 200 mg vedolizumab may be administered by intravenous infusion at six weeks, and then at one-, two-, three-, four-, five-, six-, seven-, eight-, nine-, or ten week intervals thereafter, vedolizumab may be administered subcutaneously, e.g., at a dose of 54, 108, 165, or 216 mg. In some embodiments, the subcutaneous dose is 54 mg. In other embodiments, the subcutaneous dose is 108 mg.

In some embodiments, 100 or 200 mg vedolizumab may be administered by intravenous infusion to a patient weighing less than 30 kg, or 10 kg to less than 30 kg at zero and two weeks, 54, 108, 165, or 216 mg vedolizumab may be administered subcutaneously at six weeks, and then at one-, two-, three-, four-, five-, six-, seven-, eight-, nine-, or ten-week intervals thereafter, vedolizumab may be administered subcutaneously, e.g., at a dose of 54, 108, 165, or 216 mg. In some embodiments, the subcutaneous dose is 54 mg. In other embodiments, the subcutaneous dose is 108 mg.

In some embodiments, 300 mg vedolizumab may be administered by intravenous infusion to a pediatric patient weighing 30 kg or more at zero, two, and six weeks, and then at one-, two-, three-, or four-week intervals thereafter, vedolizumab may be administered subcutaneously, e.g., at a dose of 108 mg or 216 mg.

In some embodiments, 300 mg vedolizumab may be administered by intravenous infusion to a pediatric patient weighing 30 kg or more at zero and two weeks, and then at six weeks, and at one-, two-, three-, or four-week intervals thereafter, vedolizumab may be administered subcutaneously, e.g., at a dose of 108 mg or 216 mg.

The interval between subcutaneous doses may be shorter for larger pediatric patients, e.g., weighing 30 kg or more, so they receive a subcutaneous dose at 1 to 6 week intervals and longer for smaller pediatric patients e.g., weighing less than 30 kg, or 10 kg to less than 30 kg, so they receive a subcutaneous dose at 3 to 10 week intervals.

In some embodiments, the method of treatment, dose or dosing regimen reduces the likelihood that a patient will develop a HAHA response to the anti-α4β7 antibody. The development of HAHA, e.g., as measured by antibodies reactive to the anti-α4β7 antibody, can increase the clearance of the anti-α4β7 antibody, e.g., reduce the serum concentration of the anti-α4β7 antibody, e.g., lowering the number of anti-α4β7 antibody bound to α4β7 integrin, thus making the treatment less effective. In some embodiments, to prevent HAHA, the patient can be treated with an induction regimen followed by a maintenance regimen. In some embodiments, there is no break between the induction regimen and the maintenance regimen. In some embodiments, the induction regimen comprises administering a plurality of doses of anti-α4β7 antibody to the patient. To prevent HAHA, the patient can be treated with a high initial dose, e.g., at least 1.5 mg/kg, at least 2 mg/kg, at least 2.5 mg/kg, at least 3 mg/kg, at least 5 mg/kg, at least 8 mg/kg, at least 10 mg/kg, about 5 to 25 mg/kg, about 6 to 20 mg/kg, or about 2 to about 6 mg/kg, or frequent initial administrations, e.g., about once per week, about once every two weeks or about once every three weeks, of the standard dose when beginning therapy with an anti-α4β7 antibody. In some embodiments, the method of treatment maintains at least 30%, at least 40%, at least 50%, at least 60%, at least 70%, at least 80%, at least 90% or at least 95% of patients as HAHA-negative. In other embodiments, the method of treatment maintains patients as HAHA-negative for at least 6 weeks, at least 10 weeks at least 15 weeks, at least six months, at least 1 year, at least 2 years, or for the duration of therapy. In some embodiments, the patients, or at least 30%, at least 40%, at least 50% or at least 60% of patients who develop HAHA maintain a low titer, e.g., ≤125, of anti-α4β7 antibody. In an embodiment, the method of treatment maintains at least 70% of patients as HAHA-negative for at least 12 weeks after beginning therapy with an anti-α4β7 antibody.

The dose of anti-α4β7 antibody may be administered to an individual (e.g., a human) alone or in conjunction with another agent. A dose can be administered before, along with or subsequent to administration of the additional agent. In one embodiment, more than one formulation which inhibits the binding of α4β7 integrin to its ligands is administered. In such an embodiment, an agent, e.g., a monoclonal antibody, such as an anti-MAdCAM (e.g., anti-MAdCAM-1) or an anti-VCAM-1 monoclonal antibody can be administered. In another embodiment, the additional agent inhibits the binding of leukocytes to an endothelial ligand in a pathway different from the α4β7 pathway. Such an agent can inhibit the binding, e.g. of chemokine (C-C motif) receptor 9 (CCR9)-expressing lymphocytes to thymus expressed chemokine (TECK or CCL25) or an agent which prevents the binding of LFA-1 to intercellular adhesion molecule (ICAM). For example, an anti-TECK or anti-CCR9 antibody or a small molecule CCR9 inhibitor, such as inhibitors disclosed in PCT publication WO03/099773 or WO04/046092, or anti-ICAM-1 antibody or an oligonucleotide which prevents expression of ICAM, is administered in addition to a formulation of the present invention. In yet another embodiment, an additional active ingredient (e.g., an anti-inflammatory compound, such as sulfasalazine, azathioprine, methotrexate, 6-mercaptopurine, 5-aminosalicylic acid containing anti-inflammatories, another non-steroidal anti-inflammatory compound, a steroidal anti-inflammatory compound, or antibiotics commonly administered for control of IBD (e.g. ciprofloxacin, metronidazole), probiotics, or another biologic agent (e.g. TNF alpha antagonists) can be administered in conjunction with a formulation of the present invention.

In an embodiment, the dose of the co-administered medication can be decreased over time during the period of treatment with the anti-α4β7 antibody. For example, a patient being treated with a steroid (e.g. prednisone, prednisolone, budesonide) at the beginning, or prior to, treating with the anti-α4β7 antibody would undergo a regimen of decreasing doses of steroid beginning as early as 2 weeks or 6 weeks of treatment with the anti-α4β7 antibody formulation. The steroid dose will be reduced by about 25% within 4-8 weeks of initiating tapering, by 50% at about 8-12 weeks and 75% at about 12-16 weeks of tapering during treatment with the anti-α4β7 antibody formulation. In one aspect, by about 16-24 weeks of treatment with the anti-α4β7 antibody, the steroid dose can be eliminated. In another example, a patient being treated with an anti-inflammatory compound, such as 6-mercaptopurine at the beginning, or prior to, treating with the anti-α4β7 antibody formulation would undergo a regimen of decreasing doses of anti-inflammatory compound similar to the tapering regimen for steroid dosing as noted above. In other embodiments, a corticosteroid dose of >20 mg/day may be tapered by 5 mg/week down to 20 mg/day for pediatric patients 40 kg or more, or down to 0.5 mg/day for pediatric patients less than 40 kg. In other embodiments, corticosteroid dose of <20 mg/day may be tapered by 5 mg/week down to 10 mg/day for pediatric patients 40 kg or more, or down to 0.25 mg/day for pediatric patients less than 40 kg. In some embodiments, between 6 and 14 weeks of treatment with the anti-α4β7 antibody, the corticosteroid may be further tapered by 5 mg/wk down to 10 mg/day then by 2.5 mg/week down to zero corticosteroid.

The dose of anti-α4β7 antibody, e.g., by intravenous infusion, can be administered to the pediatric patient in about 20 minutes, about 25 minutes, about 30 minutes, about 35 minutes, about 40 minutes, about 60 minutes, about 90 minutes, or about 120 minutes. In some embodiments, for a pediatric patient weighing 20 kg or higher, the infusion time is about 30 to 60 minutes. The administration may be slower for a pediatric patient having low weight (e.g., less than 20 kg). In some embodiments, for a pediatric patient weighing less than 20 kg, the infusion time is about 2 hours.

The dosing regimen can be optimized to induce a clinical response and clinical remission in the inflammatory bowel disease of the patient. In some embodiments, the pediatric patient suffering from UC achieves a clinical response based on the complete Mayo score by week 6, week 8, week 10, week 12, week 14 or week 22 after beginning treatment with the anti-α4β7 antibody. In some embodiments, the pediatric patient suffering from CD achieves a clinical response based on the CDAI score by week 6, week 8, week 10, week 12, week 14 or week 22 after beginning treatment with the anti-α4β7 antibody. In some embodiments, the UC pediatric patient achieves a clinical response of a 20 point or greater decrease from Baseline in the PUCAI score and/or a clinical remission of a PUCAI score of less than 10 by week 6, week 8, week 10, week 12, week 14 or week 22, after beginning treatment with the anti-α4β7 antibody. In some embodiments, the CD pediatric patient achieves a clinical response of a 15 point or greater decrease from Baseline in the PCDAI score with a total PCDAI of 30 or less and/or a clinical remission of a PCDAI score of 10 or less by week 6, week 8, week 10, week 12, week 14 or week 22, after beginning treatment with the anti-α4β7 antibody. In some embodiments, a measure of remission for CD pediatric patients is based on the CDAI components of abdominal pain, e.g., score of 1 or less for the prior 7 days, stool frequency, e.g., ten or fewer stools for the prior 7 days, and SES-CD score for endoscopy, e.g., less than 4, at least a 2-point reduction from baseline and no subscore greater than 1 in any individual variable.

In some embodiments, the use of an anti-α4β7 antibody for treatment of the pediatric patient suffering from IBD improves the growth of the patient. For example, a patient may have an increase from baseline in height, weight and or body mass index. In another example, as determined by the Tanner staging system, a measure of a clinical response by the pediatric patient to treatment by an anti-α4β7 antibody may be achievement of Tanner stage V (Marshall and Tanner, Arch. Dis. Child. 44:291-303 (1969) Marshall and Tanner, Arch. Dis. Child. 45:13-23 (1970)) by 16 years of age (female patient) or by 17 years of age (male patient). In some embodiments, the use of an anti-α4β7 antibody for treatment of the pediatric patient suffering from IBD results in mucosal healing. In some embodiments, the use of an anti-α4β7 antibody for treatment of the pediatric patient suffering from IBD reduces or eliminates the need for hospitalization and/or surgical resection of the affected mucosal tissue, such as the colon or rectum. In some embodiments, the corticosteroid use of an anti-α4β7 antibody for treatment of the pediatric patient suffering from IBD is reduced until discontinuation by week 48 of treatment described herein. In some embodiments, the use of an anti-α4β7 antibody for treatment of the pediatric patient suffering from CD provides fistula healing. In some embodiments, the dosing regimen does not alter the ratio of CD4 to CD8 in cerebrospinal fluid of patients receiving treatment.

In some aspects, a durable clinical remission, for example, a clinical remission which is sustained through at least two, at least three, at least four visits with a caretaking physician within a six month or one year period after beginning treatment, may be achieved with an optimized dosing regimen.

In some aspects, a durable clinical response, for example, a clinical response which is sustained for at least 6 months, at least 9 months, at least a year, after the start of treatment, may be achieved with an optimized dosing regimen.

The method may further comprise measurement of patient body weight. Body weight may be determined prior to treatment with the anti-α4β7 antibody, e.g., vedolizumab, i.e., at baseline, or may be measured at other times during treatment, e.g., when monitoring patient response. In one aspect, the present invention provides a method for treating IBD, e.g., ulcerative colitis or Crohn's disease, in a high weight pediatric patient with a higher dose (e.g., 150 mg, 300 mg) of an anti-α4β7 antibody, e.g., vedolizumab. In one aspect, the present invention provides a method for treating IBD, e.g., ulcerative colitis or Crohn's disease, in a low weight pediatric patient with a lower dose (e.g., 100 mg, 200 mg) of an anti-α4β7 antibody, e.g., vedolizumab.

The pediatric patient may have had a lack of an adequate response with, loss of response to, or was intolerant to treatment with 5-aminosalicylic acid, or a derivative thereof, an immunomodulator, a TNF-α antagonist, a corticosteroid or combinations thereof. The pediatric patient may not have received treatment with a TNF-α antagonist prior to treatment as described herein, e.g., with an anti-α4β7 antibody. The pediatric patient may have previously received treatment with and had an inadequate response or loss of response to at least one corticosteroid (e.g., prednisone or budesonide) for the inflammatory bowel disease. An inadequate response to corticosteroids refers to signs and symptoms of persistently active disease despite a history of at least one 4-week induction regimen that included a dose equivalent to prednisone 30 mg daily orally for 2 weeks or intravenously for 1 week. A loss of response to corticosteroids refers to two failed attempts to taper corticosteroids to below a dose equivalent to prednisone 10 mg daily orally. Intolerance of corticosteroids includes a history of Cushing's syndrome, osteopenia/osteoporosis, hyperglycemia, insomnia and/or infection.

The pediatric patient may have had a lack of an adequate response with, loss of response to, or was intolerant to treatment with an immunomodulator. An immunomodulator may be, for example, oral azathioprine, 6-mercaptopurine, or methotrexate. An inadequate response to an immunomodulator refers to signs and symptoms of persistently active disease despite a history of at least one 8 week regimen or oral azathioprine (≥1.5 mg/kg), 6-mercaptopurine (≥0.75 mg/kg), or methotrexate (≥12.5 mg/week). Intolerance of an immunomodulator includes, but is not limited to, nausea/vomiting, abdominal pain, pancreatitis, LFT abnormalities, lymphopenia, TPMT genetic mutation and/or infection.

In one aspect, the subject may have had a lack of an adequate response with, loss of response to, or was intolerant to treatment a TNF-α antagonist. A TNF-α antagonist is, for example, an agent that inhibits the biological activity of TNF-α, and preferably binds TNF-α, such as a monoclonal antibody, e.g., REMICADE (infliximab), HUMIRA (adalimumab), CIMZIA (certolizumab pegol), SIMPONI (golimumab) or a circulating receptor fusion protein such as ENBREL (etanercept). An inadequate response to a TNF-α antagonist refers to signs and symptoms of persistently active disease despite a history of at least one 4 week induction regimen of infliximab 5 mg/kg IV, 2 doses at least 2 weeks apart; one 80 mg subcutaneous dose of adalimumab, followed by one 40 mg dose at least two weeks apart; or 400 mg subcutaneously of certolizumab pegol, 2 doses at least 2 weeks apart. A loss of response to a TNF-α antagonist refers to recurrence of symptoms during maintenance dosing following prior clinical benefit. Intolerance of a TNF-α antagonist includes, but is not limited to infusion related reaction, demyelination, congestive heart failure, and/or infection.

A loss of maintenance of remission, as used herein for ulcerative colitis subjects, refers to an increase in Mayo score of at least 3 points and a Modified Baron Score of at least 2.

The methods described above with respect to treating a pediatric subject having IBD also apply to methods for treating with an α4β7-integrin antagonist, such as an anti-α4β7 antibody, e.g., vedolizumab, a pediatric patient at risk for GvHD, a pediatric patient having GvHD, a pediatric patient with a monogenic defect with IBD-like pathology, a pediatric patient with glycogen storage disease type 1b, a pediatric patient with colitis related to loss of function of IL10 and mutations in IL10 or IL10 receptors, a pediatric patient having X-linked lymphoproliferative syndrome 2 (defect in the XIAP gene), a pediatric patient having IPEX syndrome caused by mutations in the transcription factor FOXP3, a pediatric patient with very early onset inflammatory bowel disease (onset <6 years of age), a pediatric patient with indeterminate colitis (IBDU) and a pediatric patient with chronic granulomatous associated colitis. Alterations to the method of treatment for pediatric GvHD patients are described in detail below.

### Treatment of Pediatric Subjects for Graft Versus Host Disease (GvHD) Using an α4β7 Antibody

In one aspect, the invention relates to a method of treating a pediatric patient at risk of suffering from GvHD, comprising the steps of a. conditioning the immune system of the patient for hematopoietic stem cell transplant, b. administering an anti-α4β7 antibody, e.g., a humanized antibody having binding specificity for human α4β7 integrin, e.g., at a dose of 100 mg or 200 mg for pediatric patients less than 30 kg or at a dose of 150 mg or 300 mg for pediatric patients of 30 kg or more, c. waiting at least 12 hours, d. administering allogeneic hematopoietic stem cells, e. waiting thirteen days, then administering a second dose of the anti-α4β7 antibody, and f. waiting four weeks, then administering a third dose of the anti-α4β7 antibody.

In another aspect, the invention relates to a method of suppressing an immune response in a pediatric cancer patient, wherein the method comprises the step of: administering to a human patient undergoing allogeneic hematopoietic stem cell transplantation (allo-HSCT), an anti-α4β7 antibody, e.g., a humanized antibody having binding specificity for human α4β7 integrin, wherein the antibody is administered to the patient according to the following dosing regimen: a. an initial dose of 100 or 200 mg for pediatric patients less than 30 kg or at a dose of 150 mg or 300 mg for pediatric patients of 30 kg or more, of the antibody as an intravenous infusion the day before allo-HSCT; b. followed by a second subsequent dose of 100 or 200 mg for pediatric patients less than 30 kg or at dose of 150 mg or 300 mg for pediatric patients of 30 kg or more, of the antibody as an intravenous infusion at about two weeks after the initial dose; c. followed by a third subsequent dose of 100 or 200 mg for pediatric patients less than 30 kg or at a dose of 150 mg or 300 mg for pediatric patients of 30 kg or more, of the antibody as an intravenous infusion at about six weeks after the initial dose. In another aspect, the invention relates to a method of treating a pediatric patient suffering from GvHD, e.g., acute GvHD occurring after allogeneic hematopoietic stem cell transplant, using an α4β7-integrin antagonist, such as an anti-α4β7 antibody, e.g., vedolizumab. In some embodiments, the pediatric patient is administered an anti-α4β7 antibody, e.g., a humanized antibody having binding specificity for human α4β7 integrin, wherein the antibody is administered to the patient according to the following dosing regimen: a. an initial dose of 100 or 200 mg for pediatric patients less than 30 kg, or at a dose of 150 mg or 300 mg for pediatric patients of 30 kg or more, followed by another dose two weeks later, a third dose six weeks after the initial dose, a fourth dose ten weeks after the initial dose, and a fifth dose fourteen weeks after the initial dose.

In some embodiments, after the doses related to GvHD above, further treatment of a pediatric patient, e.g., for six months to a year, with 100 or 200 mg for pediatric patients less than 30 kg, or at a dose of 150 mg or 300 mg for pediatric patients of 30 kg or more, may maintain GvHD inhibition. In some embodiments, the maintenance of GvHD inhibition may use subcutaneous dosing of the pediatric patient at 54 mg, 108 mg, 160 mg, 165 mg, 216 mg or 250 mg of anti-α4β7 antibody, every 1 to 10 weeks.

### Pharmacokinetic and Pharmacodynamic Assays

The anti-α4β7 antibody, e.g., vedolizumab, concentration may be measured by any appropriate means known by those skilled in the art. In one aspect, the vedolizumab concentration is measured by a sandwich enzyme-linked immunosorbent assay (ELISA) assay. In another aspect, use of a pharmacodynamic assay, inhibition of MAdCAM-1-Fc binding to α₄β₇-expressing peripheral blood cells by the anti-α4β7 antibody, e.g., vedolizumab in the blood is used as a measure of the extent of α₄β₇ saturation by the anti-α4β7 antibody, e.g., vedolizumab.

In an embodiment, the anti-α4β7 antibody amount, e.g., in serum can be measured in a pharmacokinetic assay. An immobilized phase, such as a microtiter plate, vessel or bead is coated with a reagent which specifically binds to the anti-α4β7 antibody. The immobilized reagent is contacted with a patient sample, e.g., serum, which may or may not comprise the anti-α4β7 antibody. After incubation and washing, the anti-α4β7 antibody complexed to the coating reagent is contacted with a reagent which binds to the captured antibody and may be detected, e.g., using a label such as horseradish peroxidase (HRP). The binding reagent may be an anti-human antibody, e.g., polyclonal or monoclonal, which binds to the Fc portion of the anti-α4β7 antibody. Addition of an HRP substrate, such as 3,3′,5,5′-tetramethylbenzidine (TMB), can allow signal accumulation, such as color development, that can be measured, e.g., spectrophotographically.

In some embodiments, the coating reagent is an anti-idiotypic antibody which specifically binds to the anti-α4β7 antibody, e.g., its variable region or a portion thereof comprising one or more CDRs, such as heavy chain CDR3, SEQ ID NO:6. The anti-idiotypic anti-α4β7 antibody for use in the assay can be specific for, and thus bind, the α4β7 integrin-binding portion of the anti-α4β7 antibody but is not specific for the Fc portion of the anti-α4β7 antibody and thus does not bind the Fc portion of the anti-α4β7 antibody. The anti-idiotypic anti-α4β7 antibody for use in the assay can be specific for, and thus bind, a variable region of the heavy and/or light chain of anti-α4β7 antibody, e.g., selected from the group consisting of amino acids 20 to 140 of SEQ ID NO:1, amino acids 20 to 131 of SEQ ID NO:2 and amino acids 21 to 132 of SEQ ID NO:3. The anti-idiotypic anti-α4β7 antibody for use in the assay can be specific for, and thus bind, an antigen-binding fragment of the anti-α4β7 antibody. The anti-idiotypic antibody can be isolated from an immunization process using the anti-α4β7 antibody or an α4β7 integrin-binding portion thereof, such as an antibody fragment comprising one or more CDRs, and used as isolated or produced by a recombinant method. In some embodiments, the anti-idiotypic anti-α4β7 antibody is raised against an immunogen comprising heavy chain CDR3, SEQ ID NO:6. In other embodiments, the anti-idiotypic anti-α4β7 antibody is raised against an immunogen comprising a variable region of the heavy and/or light chain of anti-α4β7 antibody, e.g., selected from the group consisting of amino acids 20 to 140 of SEQ ID NO:1, amino acids 20 to 131 of SEQ ID NO:2 and amino acids 21 to 132 of SEQ ID NO:3. In some embodiments, the anti-idiotypic antibody is a monoclonal antibody. In some embodiments, an scFv fragment of the anti-idiotypic antibody is used in the assay. In other embodiments, the intact anti-idiotypic antibody is used in the assay.

Generation of an anti-idiotypic anti-α4β7 antibody can proceed in the following general methods. Immunization of a suitable animal (e.g., mouse, rat, rabbit or sheep) with protein, e.g., anti-α4β7 antibody or an α4β7 integrin binding portion thereof, or fusion protein comprising the portion, can be performed with the immunogen prepared for injection in a manner to induce a response, e.g., with adjuvant, e.g., complete Freund's adjuvant. Other suitable adjuvants include TITERMAX GOLD® adjuvant (CYTRX Corporation, Los Angeles, Calif.) and alum. Small peptide immunogens, such as a fragment comprising a CDR, such as CDR3 of the heavy chain can be linked to a larger molecule, such as keyhole limpet hemocyanin. Mice can be injected in a number of manners, e.g., subcutaneous, intravenous or intramuscular at a number of sites, e.g., in the peritoneum (i.p.), base of the tail, or foot pad, or a combination of sites, e.g., i.p. and base of tail. Booster injections can include the same or a different immunogen and can additionally include adjuvant, e.g., incomplete Freund's adjuvant. Generally, where a monoclonal antibody is desired, a hybridoma is produced by fusing a suitable cell from an immortal cell line (e.g., a myeloma cell line such as SP2/0, P3X63Ag8.653 or a heteromyeloma) with antibody-producing cells. Antibody-producing cells can be obtained from the peripheral blood or, preferably the spleen or lymph nodes, of animals immunized with the antigen of interest. Cells that produce antibodies can be produced using suitable methods, for example, fusion of a human antibody-producing cell and a heteromyeloma or trioma, or immortalization of an activated human B cell via infection with Epstein Barr virus. (See, e.g., U.S. Pat. No. 6,197,582 (Trakht); Niedbala et al., Hybridoma, 17:299-304 (1998); Zanella et al., J Immunol Methods, 156:205-215 (1992); Gustafsson et al., Hum Antibodies Hybridomas, 2:26-32 (1991).) The fused or immortalized antibody-producing cells (hybridomas) can be isolated using selective culture conditions, and cloned by limiting dilution. Cells which produce antibodies with the desired specificity can be identified using a suitable assay (e.g., ELISA (e.g., with immunogen immobilized on the microtiter well).

The anti-α4β7 antibody or the anti-idiotypic anti-α4β7 antibody may be produced by expression of nucleic acid sequences encoding each chain in living cells, e.g., cells in culture. A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an anti-α4β7 antibody in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3, NS0 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety. In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In other embodiments, the coating reagent is a ligand of the antibody, such as MAdCAM or an α4β7 integrin-binding fragment thereof or fusion protein comprising an α4β7-integrin binding fragment of MAdCAM fused with a non-MAdCAM protein, such as an immunoglobulin G constant domain. Examples of MAdCAM reagents and fusion proteins are described in PCT publication WO9624673 and U.S. Pat. No. 7,803,904, the entire teachings of which are incorporated herein by reference.

### HAHA Assay

The human anti-anti-α4β7 antibody activity (HAHA) can be determined by detecting and/or measuring anti-drug antibodies (ADAs) or antibodies specific to the anti-α4β7 antibody (anti-vedolizumab antibodies). There are a number of options, for example, using a screening and titration assay, a confirmation assay, and a neutralizing assay. Serum samples can be measured first in the screening sample at dilutions, for example, 1:5 and 1:50. Positive samples can be confirmed for specificity, titered, and examined for the ability to neutralize anti-α4β7 antibody, e.g., vedolizumab activity.

A screening assay can use a bridging ELISA in which the plate is coated with the anti-α4β7 antibody. The immobilized anti-α4β7 antibody captures the ADA in the test sample which is bound by an anti-α4β7 antibody conjugated to biotin, which is tagged by horseradish peroxidase (HRP)-labeled streptavidin, then detected with an enzymatic substrate, such as TMB. A positive color development, e.g., as measured in a microplate reader, such as Spectramax, with analytical software, such as SOFTMAX Pro3.1.2, indicates the presence of ADAs in the sample. The assay cut point, e.g., in biotin-avidin-HRP based bridging assay, can be determined by using normal human serum samples as negative controls. The mean absorbance values of the 10 negative control serums can be added to 1.65 times the standard deviation of the negative controls to determine the cut point. Thus, the cut point can allow for approximately a 5% false positive rate. In the presence of 1 μg/mL vedolizumab, low titer responses are interfered with such that they may become undetectable, although high levels of immunogenicity are detectable at vedolizumab concentrations greater than 1 μg/mL. For example, while the standard assay sensitivity can be 0.44 ng/ml, in the presence of 0.5 μg/ml vedolizumab, the sensitivity of the assay can be 180 ng/ml. For these reasons, serum samples can be taken greater than 4 weeks, greater than 8 weeks, greater than 12 weeks or greater than 16 weeks after the final dose of anti-α4β7 antibody. With a longer time period between the prior dose and the sampling, serum drug levels typically can be below the interference level.

Another assay method uses streptavidin coated plates, biotin-labeled anti-α4β7 antibody anchored to streptavidin coated vessels, beads or microtiter plates for the immobilized side of the bridge and heavy metal, such as ruthenium, osmium or rhenium-labeled (e.g., via a sulfo tag) anti-α4β7 antibody for the other side of the bridge. The bridged complex can be built on the plate by stepwise additions and washes between or in solution, with both sides of the bridge contacting diluted serum sample, then transferred to the plate. An example of an assay using this method has a sensitivity of 3.90 ng/ml anti-anti-α4β7 antibody. Detection of the heavy metal labeled bridge complex, e.g., a ruthenium-labeled complex, by electrochemiluminescence (ECL), e.g., in a Meso Scale Discovery Sector Imager 6000 (Rockville, Md.), may be more sensitive than an HRP method and/or have higher tolerance to the amount of anti-α4β7 antibody in the serum. Thus there would not be a need to wait for a delayed sample after the serum drug level lowers. In some embodiments, pretreatment of the serum sample with acid, e.g., acetic acid or low pH glycine, to release the anti-α4β7 antibody from the patient-derived anti-anti-α4β7 antibodies prior to contacting with the bridging anti-α4β7 antibodies can reduce the interference from the drug in the serum. For example, while the standard assay sensitivity can be 3.90 ng/ml, in the presence of 5 μg/ml vedolizumab in serum, the sensitivity of the assay can be 10 ng/ml.

In an embodiment, an assay to detect anti-vedolizumab antibodies in a sample of serum from a patient comprises diluting serum by a standard dilution factor, such as 1:5, 1:25, 1:50, and/or 1:125; treating with acetic acid; combining the acid treated diluted sample with an assay composition comprising a high pH reagent, such as high concentration TRIS buffer for neutralizing the acid, a biotin-labeled vedolizumab and a ruthenium-labeled vedolizumab for a time sufficient to form a bridge with serum-derived anti-vedolizumab antibodies between the two tagged versions of vedolizumab; transferring the complexes to a streptavidin-coated plate; washing the plate so only ruthenium complexed by the antibody bridge is present. Detection of the bound ruthenium-labeled complex and measuring the sample by electrochemiluminescence in the microplate reader can be achieved by adding a read solution such as tripropylamine and applying voltage to stimulate the ruthenium label complexed to the plate via the antibody bridge.

After the initial screening assay, samples can be further tested in a confirmatory assay that uses excess unlabeled anti-α4β7 antibody to demonstrate specificity. Confirmed positive samples can be further assessed for the ability of the HAHA to neutralize the binding of the anti-α4β7 antibody, e.g., vedolizumab to cells. A competitive flow cytometry-based assay was designed to determine the ability of the immune serum to inhibit the binding of labeled vedolizumab to an α₄β₇ integrin-expressing cell line, RPMI8866, and detection by flow cytometry.

The results can indicate categories of immunogenicity status: Negative: no positive HAHA sample; Positive: at least 1 positive HAHA sample; Transiently positive: at least 1 positive HAHA sample and no consecutive positive HAHA samples; and Persistently positive: at least 2 or more consecutive positive HAHA samples. Negative patients are likely to respond to anti-α4β7 antibody and can continue being treated with the antibody. Persistently positive patients are likely to have high clearance of anti-α4β7 antibody and may not respond to anti-α4β7 antibody treatment. Positive patients may have high clearance of anti-α4β7 antibody and may not respond to anti-α4β7 antibody. Positive patients can have an additional serum sample 2, 3, 4, 5 or 6 weeks after another dose of anti-α4β7 antibody to determine if they are persistently positive or transiently positive. Transiently positive patients are likely to respond to anti-α4β7 antibody treatment and treatment of these patients can be continued.

Titers of immunogenicity levels also may be determined. Titer categories include ≥5 (low), ≥50, ≥125, ≥625 and ≥3125 (high). A patient with a high titer in a positive sample may have high clearance of anti-α4β7 antibody and may not respond to anti-α4β7 antibody treatment. A patient with a low titer in a positive sample may respond to anti-α4β7 antibody treatment.

The invention will be more fully understood by reference to the following examples. They should not, however, be construed as limiting the scope of the invention. All literature and patent citations are incorporated herein by reference.

### EXEMPLIFICATION

### Example 1

A Phase 2, randomized, double-blind, dose-ranging study involving pediatric patients (male and female, 2 to 17 years, inclusive) with moderately to severely active UC or CD will be used to evaluate the PK, efficacy, immunogenicity, safety, and tolerability of vedolizumab IV. The pediatric patients will have demonstrated an inadequate response to, loss of response to, or intolerance of at least one of the following agents: corticosteroids, immunomodulators, and/or TNF-α antagonist therapy. Approximately 80 pediatric subjects will be enrolled to ensure that 40 subjects weighing greater than or equal to 30 kg and 40 subjects weighing less than 30 kg, as well as a minimum of 36 subjects with UC and a minimum of 36 subjects with CD, will be enrolled in the study.

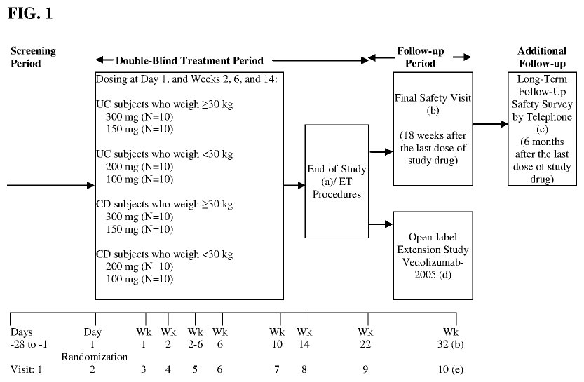

This study includes a 4-week screening period, a 22-week double blind treatment period (with last dose at week 14) for all subjects. Eligible subjects may exit the study at week 22 and continue to receive study drug in an open-label extension (OLE) study. Subjects who do not enter the OLE study will participate in an 18-week follow-up period starting from the last dose of study drug and complete a long-term follow-up safety survey by telephone six months after their last dose of study drug. A schematic of the study design is included in FIG. 1.

### Example 2

A Phase 2b, open-label, long-term extension study enrolling male and female pediatric subjects with UC or CD who initiated vedolizumab IV treatment in the Phase 2 study described in Example 1 will be done. The study will evaluate the long-term safety vedolizumab administered by IV infusion. The study will also evaluate the effect of long-term vedolizumab IV treatment on the time to major IBD-related events (hospitalizations, surgeries, or procedures), health-related quality-of-life measurements, patterns of growth and development, and exploratory efficacy measures.

Subjects will be administered vedolizumab IV once every eight weeks at the dose administered at Week 14 in the Study described in Example 1 (i.e., subjects who weigh less than 30 kg will receive 100 or 200 mg; subjects who weigh 30 kg or more will receive 150 or 300 mg). Subjects who experience disease worsening while receiving the low dose (i.e., 100 or 150 mg) may be escalated to the high dose (i.e., 200 or 300 mg) at the investigator's discretion. After completion of the study in Example 1, subjects who have their dose increased based on nonresponse should be dosed based on weight at the time of nonresponse. Blood samples will be collected every 8 weeks to assess pharmacokinetics (PK); the presence of antivedolizumab antibodies (AVA) will be assessed every 16 weeks. The study will include an 18-week Follow-up Period (Final Safety Visit) and a long-term follow-up safety survey by telephone, 6 months after the subject's last dose of study drug, for all subjects including those who discontinue the study.

### Example 3

A young monkey study was done to support the expected safety in humans. The monkeys correlate approximately to human pediatric patients (e.g., 2-4 year to 13 year old humans) and thus effects on <30 kg human patients could be inferred from this study. The objective of the study was to evaluate the toxicity and toxicokinetic profile of vedolizumab (also known as MLN0002), when administered every other week by intravenous infusion to juvenile cynomolgus monkeys for 13 weeks, as well as to evaluate the recovery, persistence or progression of any effects following a 12-week recovery period.

MLN0002 was administered once every other week by intravenous infusion (approximately 30 minutes) to juvenile cynomolgus monkeys (11 to 15 months of age and weighing between 1.2 and 2.1 kg at the start of the study) for 13 weeks in sterile water for injection as a solution at 0 (control, 0.9% physiological saline), 10, 30, and 100 mg/kg (4/sex/group). To assess the resolution of any effects, a 12-week recovery period (2/sex/group for 0 [control] and 100 mg/kg only) was conducted. The parameters evaluated were: survival, clinical observations, body weights, food consumption, ophthalmology, electrocardiology, clinical pathology parameters (hematology, coagulation, clinical chemistry, and urinalysis), toxicokinetic parameters, primate anti-human antibodies (PAHA), T-cell dependent antibody response (TDAR), flow cytometery analyses (for lymphocyte subsets in peripheral blood, cerebral spinal fluid, pharmacodynamics markers), gross necropsy findings, organ weights, and histopathologic findings.

There were no consistent gender-related differences in serum exposure to MLN0002 after dosing on Day 1 and Day 85. MLN0002 was quantifiable at the first sample collection time point post end of infusion (EOI), and median tₘₐₓ values of 0.583 hours post start of infusion (SOI), i.e., 5 minutes post EOI for all groups on both Days 1 and 85; however, tₘₐₓ values in four individuals were 24.5 and 168.5 hours post SOI (24 and 168 hours post EOI), suggesting possible extravascular dosing in those individuals.

Increases in MLN0002 dose from 10 to 30 mg/kg resulted in approximately dose proportional increases in MLN0002 AUC on Day 1. Dose proportionality of the increase in MLN0002 AUC on Day 85 at these doses could not be determined in males due to the presence of anti-MLN0002 antibodies, and was greater than dose-proportional in females (11.1-fold, n=1 female). All animals (n=4/sex) in the 10 mg/kg dose group, and 3 animals in the 30 mg/kg dose group (n=4/sex) were positive for anti-MLN0002 antibodies at 168 hours post end-of-infusion (EOI) on Day 85. The detection of antibodies in these animals was associated with a marked decrease in MLN0002 exposure at the 10 mg/kg dose, and in two of the three 30 mg/kg animals positive for anti-MLN0002 antibodies; yet, the exposure in the third 30 mg/kg animal positive for antibodies was similar to exposure in the remaining animals in the group that were negative for antibodies. Increases in MLN0002 from 30 to 100 mg/kg resulted in approximately (males) or greater (females) than dose proportional increases in MLN0002 AUC on Day 1 and Day 85, respectively.

TABLE 1
Summary of Mean Toxicokinetic Parameters of MLN002 in
Serum after Intravenous Infusion Every Other Week to
Juvenile Cynomolgus Monkeys for 13 Weeks (Excluding Animals
with Exposure Affected by Anti-MLN0002 Antibodies)

| | Tₘₐₓ^{a} | | Cₘₐₓ | | AUC_{0-168 hr} | |
|---|---|---|---|---|---|---|
| Dose | (hr) | | (μg/mL) | | (hr*μg/mL) | |
| (mg/kg) | Male | Female | Male | Female | Male | Female |
| Day 1: | | | | | | |
| 0 | N/A | N/A | <LLOQ | <LLOQ | <LLOQ | <LLOQ |
| 10 | 0.583 | 0.583 | 253 | 286 | 22,270 | 22,300 |
| 30 | 0.583 | 0.583 | 712 | 675 | 66,100 | 56,600 |
| 100 | 0.583 | 0.583 | 2460 | 3370 | 209000 | 259,000 |
| Day 85^{b}: | | | | | | |
| 0 | N/A | N/A | <LLOQ | <LLOQ | <LLOQ | <LLOQ |
| 10 | 0.583 | 0.583 | 7.87 | 41.3 | ND | ND |
| 30 | 0.583 | 0.583 | 1090 | 754 | 114,000 | 51,700 |
| 100 | 0.583 | 0.583 | 3,030 | 3,710 | 311,000 | 362,000 |

N/A = not applicable; <LLOQ = below the limit of quantitation; AUC_{0-168 hr} = area under the plasma concentration-time curve from time 0 to 168 hours; Cₘₐₓ = maximum observed; ND = not determined; tₘₐₓ = time to reach Cₘₐₓ.
^{a}Time-dependent parameters were calculated using nominal times post start of infusion (SOI)
^{b}Values excludes animals that were anti-drug antibody positive.

All animals survived to the end of the study. There were no test article-related clinical observations, or effects on body weights, food consumption, ophthalmology, electrocardiology, clinical pathology parameters (hematology, coagulation, clinical chemistry, and urinalysis), T-cell dependent antibody response (TDAR), flow cytometry analyses (peripheral blood and cerebral spinal fluid), macroscopic and microscopic findings, and organ weights.

At 10, 30, and 100 mg/kg, occupancy of the α4β7 receptors on B lymphocytes and memory CD4+T-lymphocytes in the presence of MLN0002 was demonstrated during the dosing phase as there was a reduction in the median fluorescence intensity values of labeled MLN0002 as compared to group predose values and to the control group.

In conclusion, administration of MLN0002 once every other week via intravenous infusion was well tolerated in juvenile cynomolgus monkeys at levels of 10, 30, and 100 mg/kg. There were no signs of toxicity at levels up to 100 mg/kg. Thus, 100 mg/kg was considered to be the no-observed-adverse-effect level (NOAEL) in this study. The serum AUC_{0-168 hr} and Cₘₐₓ associated with the NOAEL were 311,000 and 362,000 hr*μg/mL 3030 and 3710 μg/mL in males and females, respectively.

1. A method for treating inflammatory bowel disease in a pediatric patient, comprising intravenously administering to a pediatric patient having inflammatory bowel disease (IBD): a first dose of 200 mg of an antibody that has binding specificity for human α4β7 integrin, a second dose of 200 mg of the antibody two weeks after the first dose, and a third dose of 200 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2.

2. The method of claim 1, further comprising a fourth dose of 200 mg at 14 weeks after the first dose.

3. The method of claim 1 or 2, further comprising subsequent doses of 200 mg every eight weeks thereafter.

4. The method of any one of the previous claims, wherein the heavy chain of the antibody comprises amino acids 20 to 470 of SEQ ID NO:1, and the light chain of the antibody comprises amino acids 20 to 238 of SEQ ID NO:2.

5. The method of any one of the previous claims, wherein each dose is intravenously administered as an infusion over about 120 minutes.

6. The method of any one of the previous claims, wherein the pediatric patient weighs less than 30 kg.

7. The method of any one of claims 1-5, wherein the pediatric patient weighs 10 kg to 30 kg.

8. The method of any one of the previous claims, wherein the inflammatory bowel disease is moderately to severely active Crohn's disease.

9. The method of any one of claims 1-7, wherein the inflammatory bowel disease is moderately to severely active ulcerative colitis.

10. The method of any one of the previous claims, wherein the pediatric patient had a lack of an adequate response with, lost response to, or was intolerant to a TNFα antagonist.

11. The method of any one of claims 1-9, wherein the pediatric patient had an inadequate response or loss of response to a corticosteroid.

12. The method of any one of claims 1-9, wherein the pediatric patient had an inadequate response or loss of response to an immunomodulator.

13. The method of any one of the previous claims, wherein a clinical response is achieved as measured at week 14.

14. The method of any one of the previous claims, wherein the pediatric patient achieves remission of the inflammatory bowel disease.

15. The method of any one of the previous claims, wherein the dose is obtained from a container manufactured to deliver 200 mg of the antibody.

16. A method for treating inflammatory bowel disease in a pediatric patient, comprising intravenously administering to a pediatric patient having IBD: a first dose of 100 mg of an antibody that has binding specificity for human α4β7 integrin, a second dose of 100 mg of the antibody two weeks after the first dose, and a third dose of 100 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2.

17. The method of claim 16, further comprising a fourth dose of 200 mg at 14 weeks after the first dose.

18. The method of claim 17, further comprising a fifth and subsequent dose of 200 mg every eight weeks after the fourth dose.

19. The method of any one of the previous claims, wherein the heavy chain of the antibody comprises amino acids 20 to 470 of SEQ ID NO:1, and the light chain of the antibody comprises amino acids 20 to 238 of SEQ ID NO:2.

20. The method of any one of the previous claims, wherein each dose is intravenously administered as an infusion over about 120 minutes.

21. The method of any one of claims 16-20, wherein the pediatric patient weighs less than 30 kg.

22. The method of any one of claims 16-20, wherein the pediatric patient weighs 10 kg to 30 kg.

23. The method of any one of claims 16-22, wherein the inflammatory bowel disease is moderately to severely active Crohn's disease.

24. The method of any one of claims 16-22, wherein the inflammatory bowel disease is moderately to severely active ulcerative colitis.

25. The method of any one of claims 16-24, wherein the pediatric patient had a lack of an adequate response with, lost response to, or was intolerant to a TNFα antagonist.

26. The method of any one of claims 16-24, wherein the pediatric patient had an inadequate response or loss of response to a corticosteroid.

27. The method of any one of claims 16-24, wherein the pediatric patient had an inadequate response or loss of response to an immunomodulator.

28. The method of any one of claims 16-27, wherein a clinical response is achieved as measured at week 14.

29. The method of any one of claims 16-27, wherein the pediatric patient achieves remission of the inflammatory bowel disease.

30. The method of any one of claims 16-29, wherein the dose is obtained from a container manufactured to deliver 100 mg of the antibody.

31. A method for treating inflammatory bowel disease in a pediatric patient, comprising intravenously administering to a pediatric patient having IBD: a first dose of 150 mg of an antibody that has binding specificity for human α4β7 integrin, a second dose of 150 mg of the antibody two weeks after the first dose, and a third dose of 150 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2.

32. The method of claim 31, further comprising a fourth dose of 150 mg at 14 weeks after the first dose.

33. The method of claim 31, further comprising a fourth dose of 300 mg at 14 weeks after the first dose.

34. The method of claim 32, further comprising a fifth and subsequent dose of 150 mg every eight weeks after the fourth dose.

35. The method of claim 33, further comprising a fifth and subsequent dose of 300 mg every eight weeks after the fourth dose.

36. The method of any one of claims 31-35, wherein the heavy chain of the antibody comprises amino acids 20 to 470 of SEQ ID NO:1, and the light chain of the antibody comprises amino acids 20 to 238 of SEQ ID NO:2.

37. The method of any one of claims 31-36, wherein each dose is intravenously administered as an infusion over about 30 minutes.

38. The method of any one of claims 31-37, wherein the pediatric patient weighs 30 kg or more.

39. The method of any one of claims 31-37, wherein the pediatric patient weighs 10 kg to 30 kg.

40. The method of any one of claims 31-39, wherein the inflammatory bowel disease is moderately to severely active Crohn's disease.

41. The method of any one of claims 31-39, wherein the inflammatory bowel disease is moderately to severely active ulcerative colitis.

42. The method of any one of claims 31-41, wherein the pediatric patient had a lack of an adequate response with, lost response to, or was intolerant to a TNFα antagonist.

43. The method of any one of claims 31-41, wherein the pediatric patient had an inadequate response or loss of response to a corticosteroid.

44. The method of any one of claims 31-42, wherein the pediatric patient had an inadequate response or loss of response to an immunomodulator.

45. The method of any one of claims 31-44, wherein a clinical response is achieved as measured at week 14.

46. The method of any one of claims 31-45, wherein the pediatric patient achieves remission of the inflammatory bowel disease.

47. The method of any one of claims 31-46, wherein the dose is obtained from a container manufactured to deliver 150 mg of the antibody.

48. A method for treating inflammatory bowel disease in a pediatric patient, comprising intravenously administering to a pediatric patient having IBD: a first dose of 300 mg of an antibody that has binding specificity for human α4β7 integrin, a second dose of 300 mg of the antibody two weeks after the first dose, and a third dose of 300 mg of the antibody six weeks after the first dose, wherein the antibody comprises a heavy chain variable region sequence of amino acids 20 to 140 of SEQ ID NO:1, and a light chain variable region sequence of amino acids 20 to 131 of SEQ ID NO:2.

49. The method of claim 48, further comprising a fourth dose of 300 mg at 14 weeks after the first dose.

50. The method of claim 49, further comprising a fifth and subsequent dose of 300 mg every eight weeks after the fourth dose.

51. The method of any one claims 48-50, wherein heavy chain of the antibody comprises amino acids 20 to 470 of SEQ ID NO:1, and the light chain of the antibody comprises amino acids 20 to 238 of SEQ ID NO:2.

52. The method of any one of claims 48-51, wherein each dose is intravenously administered as an infusion over about 30 minutes.

53. The method of any one of claims 48-52, wherein the pediatric patient weighs 30 kg or more.

54. The method of any one of claims 48-53, wherein the inflammatory bowel disease is moderately to severely active Crohn's disease.

55. The method of any one of claims 48-53, wherein the inflammatory bowel disease is moderately to severely active ulcerative colitis.

56. The method of any one of claims 48-55, wherein the pediatric patient had a lack of an adequate response with, lost response to, or was intolerant to a TNFα antagonist.

57. The method of any one of claims 48-55, wherein the pediatric patient had an inadequate response or loss of response to a corticosteroid.

58. The method of any one of claims 48-55, wherein the pediatric patient had an inadequate response or loss of response to an immunomodulator.

59. The method of any one of claims 48-58, wherein a clinical response is achieved as measured at week 14.

60. The method of any one of claims 48-59, wherein the pediatric patient achieves remission of the inflammatory bowel disease.

61. A method for treating inflammatory bowel disease in a pediatric patient, comprising intravenously administering to a pediatric patient having IBD: a first dose of 100 mg of an antibody that has binding specificity for human α4β7 integrin, a second dose of 100 mg of the antibody two weeks after the first dose, and a third dose of 100 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

62. A method for treating inflammatory bowel disease in a pediatric patient, comprising intravenously administering to a pediatric patient having IBD: a first dose of 200 mg of an antibody that has binding specificity for human α4β7 integrin, a second dose of 200 mg of the antibody two weeks after the first dose, and a third dose of 200 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

63. A method for treating inflammatory bowel disease in a pediatric patient, comprising intravenously administering to a pediatric patient having IBD: a first dose of 150 mg of an antibody that has binding specificity for human α4β7 integrin, a second dose of 150 mg of the antibody two weeks after the first dose, and a third dose of 150 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

64. A method for treating inflammatory bowel disease in a pediatric patient, comprising intravenously administering to a pediatric patient having IBD: a first dose of 300 mg of an antibody that has binding specificity for human α4β7 integrin, a second dose of 300 mg of the antibody two weeks after the first dose, and a third dose of 300 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

65. The method of any one of claims 61 to 64, wherein subsequent doses of the antibody are administered subcutaneously.

66. The method of claim 65, wherein each subcutaneous dose is 108 mg of antibody.

67. The method of claim 65 or 66, wherein the subcutaneous dose is administered every two or four weeks to a pediatric patient who weighs 30 kg or more.

68. The method of claim 65 or 66, wherein the subcutaneous dose is administered every three weeks, every four weeks, every five weeks, every six weeks, every seven weeks, every eight weeks, every nine weeks or every ten weeks to a pediatric patient who weighs 10 kg to 30 kg.

69. A method for treating inflammatory bowel disease (IBD) in a pediatric patient, comprising intravenously administering to a pediatric patient having IBD: a first dose of 200 mg of an antibody that has binding specificity for human α4β7 integrin, a second dose of 200 mg of the antibody two weeks after the first dose, and subcutaneously administering a third dose of 108 mg of the antibody six weeks after the first dose and subsequent doses of 108 mg of the antibody every two, three or four weeks thereafter, wherein the antibody comprises an antigen binding region of nonhuman origin and at least a portion of an antibody of human origin, wherein the antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

70. A method for treating a pediatric cancer patient undergoing allogeneic hematopoietic stem cell transplantation (allo-HSCT), comprising intravenously administering to a pediatric patient the day before allo-HSCT a first dose of 200 mg of an antibody that has binding specificity for human α4β7 integrin, a second dose of 200 mg of the antibody two weeks after the first dose, and subcutaneously administering a third dose of 108 mg of the antibody six weeks after the first dose and subsequent doses of 108 mg of the antibody every two, three or four weeks thereafter, wherein the antibody comprises an antigen binding region of nonhuman origin and at least a portion of an antibody of human origin, wherein the antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

71. A method for treating a pediatric patient with a monogenic defect with IBD-like pathology, comprising intravenously administering to the pediatric patient: a first dose of 200 mg of an antibody that has binding specificity for human α4β7 integrin, a second dose of 200 mg of the antibody two weeks after the first dose, and a third dose of 200 mg of the antibody six weeks after the first dose, wherein the antibody comprises an antigen binding region of nonhuman origin and at least a portion of an antibody of human origin, wherein the antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

72. A vial manufactured to deliver 200 mg of anti-α4β7 antibody for treating a pediatric patient.

73. The method of claim 71, wherein said monogenic defect with IBD-like pathology is selected from the group consisting of glycogen storage disease type 1b, loss of function of IL10 and mutations in IL10 or IL10 receptors, X-linked lymphoproliferative syndrome 2, IPEX syndrome caused by mutations in the transcription factor FOXP3, and chronic granulomatous disease.

74. The method of claim 71 or 73, further comprising a subsequent dose of 200 mg every eight weeks thereafter.

75. The method of claim 71 or 73, further comprising a subsequent dose of 200 mg until the pediatric patient is 30 kg or greater.

76. The method of any one of claims 1-4, 16-19, 61-63, 71, 73, and 74 further comprising raising the dose to 300 mg after the pediatric patient weighs 30 kg or more.

77. The method of any one of claims 61-64, 69, and 71 wherein said antibody is a humanized antibody.

78. The method of claim 77, wherein said humanized antibody comprises a heavy chain variable region sequence of amino acids 20 to 140 of SEQ ID NO:1, and a light chain variable region sequence of amino acids 20 to 131 of SEQ ID NO:2.
